(19) United States
(12) Patent Application Publication
(10) Pub. No.: US 2009/0208544 A1
(43) Pub. Date: Aug. 20, 2009

(54) METHOD OF OBTAINING PHYTOALEXINS

(76) Inventors: Rachid Ennamany, Villenave D'Ornon (FR); Jean-Michel Merillon, Martillac (FR)

Correspondence Address: HOVEY WILLIAMS LLP, 10801 Mastin Blvd., Suite 1000, Overland Park, KS, 66210, US

(21) Appl. No.: 12/404,679

(22) Filed: Mar. 16, 2009

Related U.S. Application Data
(63) Continuation-in-part of application No. 10/943,698, filed on Sep. 17, 2004.
Application No. 10/943,698 is a continuation-in-part of application No. PCT/IB03/01020, filed on Mar. 20, 2003.

(30) Foreign Application Priority Data
Mar. 20, 2002 (FR) 02/03423
Sep. 26, 2002 (IB) PCT/IB02/03971

(51) Int. Cl. A61K 8/97

(52) U.S. Cl. 424/401; 424/93.7

(57) ABSTRACT

Topical composition containing (a) from 0.1% to 5% of encapsulated UV-elicited dedifferentiated plant cells comprising vacuoles rich in stilbenes, and (b) cosmetically acceptable excipients.

This application is a Continuation in Part of U.S. application Ser. No. 10/943,698 filed on Sep. 17, 2004 and published on Dec. 11, 2005 under publication number US2005/0265953, which is a Continuation in Part of international Application No. PCT/IB03/01020 filed on Mar. 20, 2003 (published under number WO03/077881 on Sep. 25, 2003), and claiming the benefit of the priority of French patent application FR 02/03423 filed on Mar. 20, 2002, as well as of International Application No. PCT/IB02/03971 filed on Sep. 26, 2002 (published under number WO03/077880 filed on Sep. 25, 2003), the entire disclosures of which are incorporated herein by reference.

### BACKGROUND OF THE INVENTION

This invention relates to a composition for topical use, particularly a cosmetic composition, which is rich in metabolites produced by dedifferentiated plant cells.

The invention relates in particular to a composition containing dedifferentiated plant cells which are elicited and which are then partially or completely dried, preferably freeze-dried, and are comminuted and dispersed in said composition.

The expression “dedifferentiated plant cells” should be understood to mean any plant cell which exhibits none of the features of a particular specialised cell classification, and which is capable of living by itself and not in dependence on other cells.

Dedifferentiated plant cells can be obtained from plant material which is derived from a whole plant or from part of a plant, such as leaves, stems, flowers, petals, roots, fruit, skin, the envelope protecting them, seeds, anthers, sap, thorns, buds, peel, berries and mixtures thereof.

Dedifferentiated plant cells are preferably obtained from peel, leaves, buds and from the skin of fruit, particularly from fruit cuticles.

Dedifferentiated plant cells which can be used according to the invention can be obtained from plants obtained by in vivo culture or derived from in vivo culture.

The expression “in vivo culture” should be understood to mean any classical type of culture, i.e. in soil, in the fresh air, in a greenhouse or in a soil-free or hydroponic environment.

The expression “in vitro culture” should be understood to mean the all the techniques known to one skilled in the art which enable a plant or a part of a plant to be obtained artificially. The pressure of selection imposed by the physicochemical conditions during the growth of plant cells in vitro enables a standardised plant material to be obtained which is free from contaminants and is available all year round, in contrast to plants cultivated in vivo.

According to the invention, dedifferentiated plant cells are preferably used which are derived from culture in vitro.

The dedifferentiated plant cells which can be used according to the invention can be obtained by any method which is known from the prior art. Methods which can be cited in this respect include those described by E. F. George and P. D. Sherrington in Plantation Propagation by Tissue Culture, Handbook and Directory of Commercial Laboratories (Exegetics Ltd. 1984).

The culture media which can be used according to the invention are those which are generally known to one skilled in the art. Examples which can be cited include the media of Gamborg, Murashige and Skoogs, Heller, White etc. Complete descriptions of these media are given in “Plantation Culture Media: Formulations and Uses” by E. F. George, D. J. M. Puttock and H. J. George (Exegetics Ltd. 1987, Volumes 1 & 2).

According to the invention, the cultivated, dedifferentiated plant cells are preferably prepared on the medium of Murashige and Skoog.

### PRIOR ART

FR 2795637 discloses a cosmetic composition containing an extract of dedifferentiated plant cells to avoid odour problems. This composition contains an extract of plant cells which are dedifferentiated but not elicited, so that this composition has a low content of secondary metabolites or phytoalexins, is or even substantially free from such compounds. Moreover, this document describes the use of aqueous extracts obtained after comminution of the cells in their culture medium followed by the removal of the particles in suspension, with an unavoidable loss of metabolites bound to the particles in suspension. In order to remove proteases, and oxidases in particular, this document also recommends the use of filters which retain molecules with a molecular weight higher than 100,000 daltons, which thus results in the loss from the final extract of all metabolites with a molecular weight higher than this weight, which can prove to be of great interest to the cosmetics industry. Furthermore, in order to eliminate problems due to oxidation this document recommends the addition of stabilisers, particularly cysteine, and/or of sulphur-containing derivatives, which inevitably results in the purity of the extract being reduced during subsequent filtration stages. The methods described in this document necessitate the use of complicated means for obtaining extracts, the purity (numerous additives) and the quality and concentration (of metabolites) of which are not optimal. Moreover, the numerous stages necessary to obtain extracts by this method result in increased costs and in the risk of contamination due to the numerous manipulations and additives employed.

Cultures of dedifferentiated cells are known, as are the mechanisms of elicitation of these cells followed by extraction stages and by various filtrations followed by freeze-drying in order to incorporate the extracts obtained in a cosmetic or pharmaceutical preparation. Such methods are described, for example, in U.S. Pat. No. 4,241,536; EP 378 921, WO 88/00968, EP 1 203 811, etc. for species of various plants. The entire disclosure and content of these documents is incorporated in the present description by reference in order to describe culture media, plant species, possible elicitors, etc.

A cosmetic composition comprising dedifferentiated Gingko biloba cells or an extract of said cells has been proposed in FR2744915. Said gingko biloba cells have not been elicited and are not encapsulated at least with glycosaminoglycan.

By repeating the production of said dedifferentiated Gingko Biloba cells, it has been shown that the cells were not stable in colour, that bad odour was expelled from the cells with the time, and that the stilbene content was not as high as possible.

Furthermore, when comminuting the dried plant cells, the cells were completely destroyed, whereby the vacuole content thereof was released out of the cell membrane.

European Patent No. EP0085589 discloses a process for the preparation of stabilized fresh cell suspensions, requiring the step of grinding the fresh cell in presence of ethanol, glycerol, mono propylene-glycol, propylene glycol, ethyl diglycol, dipropylene glycol, tripropylene glycol and iso-propylidene glycerol. The amount of additive used corresponds to more than 10 times the dry weight of the fresh cells.

The so achieved fresh cells were not stable in color and odor, while having a poor stilbene content.

European Patent No. EP1392220 discloses a cosmetic product comprising plant cells encapsulated in degradable micro-particles suitable for a progressive release of the active ingredient, when the product is applied on the skin. The plant cell are encapsulated into a polysaccharide matrix, especially having glyceric cross linkage maltodextrins. The encapsulated plant cells of said document were not stable in colour and odour with the time.

U.S. Patent Publication No. US2007/0098668 discloses the use of a lyophilisate of dedifferentiated plant cells in a cosmetic composition. The so produced dedifferentiated plant cells are not encapsulated with glyosaminoglycans, nor with trenalose, whereby said cells were not stable in colour and odour with the time.

The article “anti-inflammatory activity in extracts prepared from callus cultures of Eucomis autumnalis (Mill.) Chitt.”, Taylor et al, Plant Growth Regulation 34: 331-337, 2001 discloses that high levels of anti-inflammatory activity have been detected in extracts prepared from Eucomis plants as well as from in vitro plantlets. In said article, the cells were ground in liquid nitrogen before being mixed in ethanol for extracting soluble compounds from the cells. The ethanol and solubilized compounds were recovered by filtration.

Said article does not teach, or suggest the use of encapsulated elicited plant cells in cosmetic compositions.

U.S. Pat. No. 6,414,037 relates to a pharmaceutical formulation comprising resveratrol, which can be administered as chemically synthesised or in natural form. The preferred methods for obtaining resveratrol from a natural source is the extraction of resveratrol by methanol from dried ground plant material. Said patent does not teach, or suggest the use of encapsulated elicited plant cells in cosmetic compositions.

Currently, despite the expertise and know-how of industries in the field of plant extraction, and despite the progress of organic chemistry, several extraction stages are necessary in order to obtain a plant raw material having a high stilbene content, which is stable, said plant cells having the properties of keeping the plant cell shape and/or keeping its colour for at least 3 months, advantageously at least 6 months and/or low degradation even after 6 months storage at 20° C. in a humid atmosphere.

In the composition of the invention, the encapsulated cells are integral, whereby the stilbenes, phytoalexins, polypeptides, proteins, vitamins, etc present in the cells are not in direct contact with the excipients of the cosmetic composition. The cells are broken when pressing or massaging the cosmetic composition onto the skin, whereby liberating the active agents present within the cells directly on the skin layer.

As the cells of the composition of the invention are not associated with alcohol extraction stages, the cells do not have loss of the tertiary structure of the active agent molecules present in the cells.

As in the composition of the invention, the plant cells are produced in vitro and elicited in vitro, while being encapsulated, the cells will not comprises toxic solvents (used normally for extracting one or more active agents), will have a stable and uniform quality all the year, independently from the seasons.

### SUMMARY OF THE INVENTION

The inventors have developed an innovative, controlled technology which ensures the quality and authenticity of the products. It involves placing cells of dedifferentiated higher plants in an in vitro culture, encapsulate cells and elicited cells.

In fact, and for the first time, an industrial process is proposed which enables cells to be obtained from higher plants by a method which avoids any modification of their genetic heritage, allowing the cells to retain its physiological features and to keep its physiological features for period of more than 3 months, even more than 6 months, such as for period of 1 to 5 years, without variation of colour or the appearance of some bad odour.

Maintenance of the various strains is ensured by regular subculturing, with total control of the different conditions of culture.

The importance of this method is that it enables the culture of dedifferentiated plant cells to be effected on a large scale whilst responding to the needs of the industry, in particular:
Preservation of the tertiary structure of the molecules,
Absence of solvent and residues,
Substrate homogeneity,
Continuous processing, regardless of the cycle of the seasons,
Retention of biological and physiological characteristics without the addition of preservatives,
Complete absence of pollutants,
Standardised, reproducible production with regard to metabolite quality and concentration,
The use of these plant suspensions, possibly after filtration step, after direct freeze-drying at a temperature less than 30° C. This technique enable a very fine powder to be obtained which is suitable for dispersion in cosmetic compositions (creams, ointments, lotions, etc.). These cells are capable of directly releasing the active constituents which they contain without passage through an extraction stage using organic solvents (elimination of the risk of residues). However, the product of freeze-drying is preferably subjected to comminution to prevent any agglomeration of particles, said comminution being more easy due to the encapsulation of cells.

This technology provides a useful, innovative alternative to conventional solvent extraction methods. The possibility of naturally orienting (by elicitation) the synthesis of metabolites without undermining the genetic integrity of the cells constitutes a guarantee of quality and authenticity.

### BRIEF DESCRIPTION OF THE INVENTION

The invention relates to a cosmetic composition for topical application containing at least:

(a) a dispersion of at least encapsulated UV-elicited dedifferentiated plant cells, whereby said encapsulated UV-elicited dedifferentiated plant cells comprise vacuoles comprising at least stilbenes, vitamins, proteins and amino acids, whereby the dry weight of stilbenes selected from the group consisting of resveratrol (especially in the form of trans-resveratrol), catechine (especially as catechin or epicatechin), astringine (especially as trans-astringine and cis-astringine) and piceide (especially as trans-piceide and cis-piceide) with respect to the dry weight of the encapsulated UV-elicited dedifferentiated plant cells before being communited is at least 0.1% (for example comprised between 0.1% and 1%, such as between 0.2% and 0.5%) (the cells can comprise other stilbene compounds), the cell being provided with a bi-lipidic membrane comprising at least C₁₂-C₂₀ fatty acids, cholesterol and ceramides, whereby said encapsulated UV-elicited dedifferentiated plant cells are at least partly encapsulated with glycosaminoglycans or a glycosaminoglycans containing coating and whereby said encapsulated UV-elicited dedifferentiated plant cells are uncommunited, whereby the cosmetic composition comprises from 0.1% by weight to 5% by weight of said encapsulated UV-elicited dedifferentiated plant cells, and

(b) a cosmetically acceptable excipient.

The cosmetically acceptable excipients used will be selected so as not to destroy or to attack the encapsulated plant cells, especially the glycosaminoglycans containing coating, or will be used in concentration not sufficient for destroying or to attacking the encapsulated plant cells, especially the glycosaminoglycans containing coating.

Possibly some agglomerations of elicited dedifferentiated plant cells are encapsulated with glycosaminoglycans containing coating.

Advantageously, the encapsulated UV-elicited dedifferentiated plant cells are dedifferentiated plant cells grown in suspension in vitro in a culture medium and UV-elicited in said culture medium for at least 7 to 30 days for synthesising stilbenes into the cells and for storing stilbenes within the vacuoles of the UV-elicited dedifferentiated plant cells.

During said elicitation step, the plant cells are enriched in one or more (advantageously more) components such as phytoalexins, trans-resveratrol, catechine, epicatechine, trans-astringine, cis-astringine, trans-piceide, cis-piceide, vitamins, proteins, unsaturated fatty acids, sterols.

According to an embodiment, the encapsulated UV-elicited dedifferentiated plant cells are dedifferentiated plant cells grown in suspension in vitro in a culture medium and UV-elicited in said culture medium for at least 7 to 30 days for synthesising stilbenes into the cells and for storing stilbenes within the vacuoles of the UV-elicited dedifferentiated plant cells, whereby the culture medium comprises at least one sugar, such as gelose, sucrose, saccharose, maltose, maltotriose, mixtures thereof, and at least one additive selected from the group consisting of unsaturated C₁₂-C₂₀ fatty acid (especially unsaturated C₁₂, C₁₄, C₁₆ and C₁₈ fatty acid and mixtures thereof) and pectin.

Preferably, the weight ratio additive selected from the group consisting of unsaturated C₁₂-C₂₀ fatty acid and pectin present in the culture medium/dedifferentiated plant cells growing in the culture medium expressed in dry from is comprised between 0.2:1 and 10:1, preferably between 0.5:1 and 5:1.

The encapsulated UV-elicited dedifferentiated plant cells are advantageously dedifferentiated plant cells grown in suspension in vitro in a culture medium and UV-elicited in said culture medium for at least 7 to 30 days for synthesising stilbenes into the cells and for storing stilbenes within the vacuoles of the UV-elicited dedifferentiated plant cells, whereby the culture medium comprises at least sucrose and at least one additive selected from the group consisting of unsaturated C₁₂-C₂₀ fatty acid and pectin.

Preferably, the weight ratio additive selected from the group consisting of unsaturated C₁₂-C₂₀ fatty acid and pectin present in the culture medium/dedifferentiated plant cells growing in the culture medium expressed in dry form is comprised between 0.2:1 and 10:1, preferably between 0.5:1 and 5:1.

The composition comprises advantageously from 0.2% by weight to 2% by weight of said encapsulated UV-elicited dedifferentiated plant cells. According to preferred embodiments, the composition comprises at least encapsulated UV-elicited dedifferentiated vine (as used herein, unless otherwise stated, “vine” refers to any plant of the genus Vitis (the grape plants)) cells, for example mixed with encapsulated UV-elicited dedifferentiated plant cells of one or more other species.

The composition of the invention can possibly comprise encapsulated UV-elicited dedifferentiated plant cell selected from the group species consisting of Salvia, Coleus, Rosmarinus, Ginkgo, Cannabis, Colchicum, Gloriosa, Asparagus, Arganier, Wisteria, Medicago, Mungo, Eryhrina, Oenothera, Papaver, Atropa, Datura, Solanum, Borago, Reseda, Amsonita, Catharantus, Pilocarpus, Digitalis, Coffea, Theobroma, Jasminum, Capsicum, Iris, vine, taxus, blue lotus, oriental cherry, sequoia, chlorophytum, Cacao, psoralea coryilfolia, vitex negundo, commiphora wighii, eucalyptus punctata, lavandula angustifolia, citrus limon, vanilla planifolia, marrubium vulgare, pilocarpus jaborandi, roses, betula, tea, and mixtures thereof.

According to a specific embodiments of compositions of the invention, the encapsulated UV-elicited dedifferentiated plant cells are at least partly encapsulated with glycosaminoglycans and at least one component selected from the group consisting of glycerol, ethyleneglycol, propyleneglycol, diethyleneglycol, butylene glycol and mixtures thereof (glycerol and butylene glycol and mixtures thereof being most preferred).

The weight ratio component selected from the group consisting of glycerol, ethyleneglycol, propyleneglycol, diethyleneglycol, butyleneglycol and mixtures thereof/UV-elicited dedifferentiated plant cells is advantageously greater than 2:1, preferably comprised between 3:1 and 10:1.

According to a detail of embodiments, the encapsulated UV-elicited dedifferentiated plant cells are dedifferentiated plant cells grown in suspension in vitro in a culture medium, encapsulated at least with glycosaminoglycans and at least one component selected from the group consisting of glycerol, ethyleneglycol, propyleneglycol, diethyleneglycol, butyleneglycol and mixtures thereof, before being UV-elicited in said culture medium for at least 7 to 30 days for synthesising stilbenes into the cells and for storing stilbenes within the vacuoles of the UV-elicited dedifferentiated plant cells.

According to another detail of embodiments, the encapsulated UV-elicited dedifferentiated plant cells are at least partly encapsulated with glycosaminoglycan selected from the group consisting of Chondroitin and cosmetically acceptable salts thereof, heparin and cosmetically acceptable salts thereof, Heparitin and cosmetically acceptable salts thereof, Hyaluronic Acid and cosmetically acceptable salts thereof, Keratan and cosmetically acceptable salts thereof, and their mixtures.

According to a preferred embodiment of compositions of the invention, the encapsulated UV-elicited dedifferentiated plant cells are at least partly encapsulated with glycosaminoglycans and trehalose. The weight ratio glycosaminoglycans/trehalose is advantageously comprised between 1:10 and 10:1, preferably comprised between, 1:10 and 1:1.

According to a specific embodiment, the composition further comprises powder of communited lyophilized encapsulated UV-elicited dedifferentiated plant cells, whereby said encapsulated UV-elicited dedifferentiated plant cells comprise, before being communited, vacuoles comprising at least stilbenes, vitamins, proteins and amino aids, whereby the dry weight of stilbenes selected from the group consisting of resveratrol, catechine, astringine and piceide with respect to the dry weight of the encapsulated UV-elicited dedifferentiated plant cells before being communited is at least 0.1%, the encapsulated UV-elicited dedifferentiated plant cells before being communited being provided with a bi-lipidic membrane comprising at least C₁₂-C₂₀ fatty acids cholesterol and ceramides, whereby said encapsulated UV-elicited dedifferentiated plant cells before being communited are at least partly encapsulated with glycosaminoglycans. The dry weight ratio powder of communited lyophilised encapsulated UV-elicited dedifferentiated plant cells/non communited encapsulated UV-elicited dedifferentiated plant cells is advantageously comprised between 1:10 and 10:1, preferably between 1.5 and 5:1.

Preferably, the powder of communited lyophilised encapsulated UV-elicited dedifferentiated plant cells is prepared from dedifferentiated plant cells at least partly encapsulated with glycosaminoglycan selected from the group consisting of Chondroitin and cosmetically acceptable salts thereof, heparin and cosmetically acceptable salts thereof. Heparitin and cosmetically acceptable salts thereof. Hyaluronic Acid and cosmetically acceptable salts thereof, Keratan and cosmetically acceptable salts thereof; and their mixtures.

Preferably, the powder of communited lyophilized encapsulated UV-elicited dedifferentiated plant cells is a powder of communited UV-elicited dedifferentiated plant cells encapsulated at least with glycosaminoglycans and trehalose. The weight ratio glycosaminoglycans/trehalose is advantageously comprised between 1:10 and 10:1, preferably comprised between, 1:10 and 1:1.

The invention relates also to a cosmetic composition for topical application containing at least:

(a) a powder of communited lyophilized encapsulated UV-elicited dedifferentiated plant cells, whereby said encapsulated UV-elicited dedifferentiated plant cells comprise before being communited vacuoles comprising at least stilbenes, vitamins, proteins and amino acids, whereby the dry weight of stilbenes selected from the group consisting of resveratrol (especially in the form of trans-resveratrol), catechine (especially as catechin or epicatechin), astringine (especially as trans-astringine and cis-astringine) and piceide (especially as trans-piceide and cis-piceide) with respect to the dry weight of the encapsulated UV-elicited dedifferentiated plant cells before being communited is at least 0.1% (for example comprised between 0.1% and 1% such as between 0.2% and 0.5%) (the cells can comprise other stilbene compounds), the encapsulated UV-elicited dedifferentiated plant cells before being communited being provided with a bi-lipidic membrane comprising at least C₁₂-C₂₀ fatty acids, cholesterol and ceramides, whereby said encapsulated UV-elicited dedifferentiated plant cells before being communited are at least partly encapsulated with glycosaminoglycans, whereby the cosmetic composition comprises from 0.1% by weight to 5% by weight of said powder of communited lyophilized encapsulated UV-elicited dedifferentiated plant cells, and

(b) a cosmetically acceptable excipient.

The cosmetically acceptable excipient(s) used will be selected so as not to destroy or to attack the encapsulated plant cells in powder form, especially the glycosaminoglycans containing coating, or will be used in concentration not sufficient for destroying or to attacking the encapsulated plant cells in powder form, especially the glycosaminoglycans containing coating.

Possibly some agglomerations of powder of elicited dedifferentiated plant cells are encapsulated with glycosaminoglycans containing coating.

According to an embodiment, said powder of comminuted lyophilised encapsulated UV-elicited dedifferentiated plant cells is prepared from dedifferentiated plant cells at least partly encapsulated with glycosaminoglycan selected from the group consisting of Chondroitin and cosmetically acceptable salts thereof, heparin and cosmetically acceptable salts thereof, Heparitin and cosmetically acceptable salts thereof, Hyaluronic Acid and cosmetically acceptable salts thereof, Keratan and cosmetically acceptable salts thereof, and their mixture.

The powder has advantageously an average particle size of less than 10 μm, preferably of less than 1 μm.

The communition of the dried plant cells aggregates will be operated in a mildly manner so as not to break completely the encapsulating layer covering the plant cells, i.e. so that after communition, the plant cells in powder form are still encapsulated.

The composition advantageously comprises powder of communited lyophilised encapsulated UV-elicited dedifferentiated vine cells. The composition can however also comprise powder of comminuted lyophilised encapsulated UV-elicited dedifferentiated plant cells of one or more different species.

According to a possible embodiment, the powder of comminuted lyophilised encapsulated UV-elicited dedifferentiated cells is derived from the culture of dedifferentiated plant cells, which are elicited and then dried, of at least one species selected from the group consisting of Salvia, Coleus, Rosmarinus, Gingko, Cannabis, Colchicum, Gloriosa, Asparagus, Arganier, Wisteria, Medicago, Mungo, Erythrina, Oenothera, Papaver, Atropa, Datura, Solanum, Borago, Reseda, Amsonia, Catharantus, Pilocarpus, Digitalis, Coffea, Theobroma, Jasminum, Capsicum, Iris, vine, taxus, blue lotus, oriental cherry, sequoia, chlorophytum, Cacao, psoralea coryilfolia, vitex negundo, commiphora wighii, eucalyptus punctata, lavandula angustifolia, citrus limon, vanilla planifolia, marrubium vulgare, pilocarpus jaborandi, roses, betula, tea and mixtures of cells of such species.

According to a preferred embodiment, the powder of communited lyophilized encapsulated UV-elicited dedifferentiated plant cells is a powder of communited UV-elicited dedifferentiated plant cells encapsulated at least with glycosaminoglycans and trehalose. The weight ratio glycosaminoglycans/trehalose is advantageously comprised between 1:10 and 10:1, preferably comprised between 1:10 and 1:1.

According to a specific embodiments of compositions of the invention the encapsulated UV-elicited dedifferentiated plant cells in powder form are at least partly encapsulated with glycosaminoglycans and at least one component selected from the group consisting of glycerol, ethyleneglycol, propyleneglycol, diethyleneglycol, butylene glycol and mixtures thereof (glycerol and butylene glycol and mixtures thereof being most preferred).

The weight ratio component selected from the group consisting of glycerol, ethyleneglycol, propyleneglycol, diethyleneglycol, butyleneglycol and mixtures thereof/UV-elicited dedifferentiated plant cells is advantageously greater than 2:1, preferably comprised between 3:1 and 10:1.

According to a detail of embodiments, the encapsulated UV-elicited dedifferentiated plant cells in powder form are dedifferentiated plant cells grown in suspension in vitro in a culture medium, encapsulated at least with glycosaminoglycans and at least one component selected from the group consisting of glycerol, ethyleneglycol, propyleneglycol, diethyleneglycol, butyleneglycol and mixtures thereof, before being UV-elicited in said culture medium for at least 7 to 30 days for synthesising stilbene into the cells and for storing stilbene within the vacuoles of the UV-elicited dedifferentiated plant cells.

According to another detail of embodiments, the encapsulated UV-elicited dedifferentiated plant cells in powder form are at least partly encapsulated with glycosaminoglycan selected from the group consisting of Chondroitin and cosmetically acceptable salts thereof, heparin and cosmetically acceptable salts thereof, Heparitin and cosmetically acceptable salts thereof, Hyaluronic Acid and cosmetically acceptable salts thereof, Keratan and cosmetically acceptable salts thereof, and their mixtures.

The invention further relates to a method of preparing a cosmetic composition for topical application containing at least;

(a) a dispersion of at least encapsulated UV-elicited dedifferentiated plant cells, whereby said encapsulated UV-elicited dedifferentiated plant cells comprise vacuoles comprising at least stilbenes, vitamins, proteins and amino acids, whereby the dry weight of stilbenes selected from the group consisting of resveratrol (especially in the form of trans-resveratrol), catechine (especially as catechin or epicatechin), astringine (especially as trans-astringine and cis-astringine) and piceide (especially as trans-piceide and cis-piceide) with respect to the dry weight of the encapsulated UV-elicited dedifferentiated plant cells before being communited is at least 0.1% (for example comprised between 0.1% and 1%, such as between 0.2% and 0.5%) (the cells can comprise other stilbene compounds), the cell being provided with a bi-lipidic membrane comprising at least C₁₂-C₂₀ fatty acids, cholesterol and ceramides, whereby said encapsulated UV-elicited dedifferentiated plant cells are at least partly encapsulated with glycosaminoglycans and whereby said encapsulated UV-elicited dedifferentiated plant cells are uncommunited, whereby the cosmetic composition comprises from 0.1% by weight to 5% by weight of said encapsulated UV-elicited dedifferentiated plant cells, and

(b) a cosmetically acceptable excipient,

said method comprising at least the following steps:
step 1: dedifferentiated plant cells are growth in vitro in an agar-agar containing culture medium in a sterile atmosphere at a temperature comprised between 10° C. and 35° C., so as to form a culture of dedifferentiated plant cells;
step 2: dedifferentiated plant cells from the culture of step 1 are put in suspension and mixed in a liquid sucrose containing culture medium enriched with at least 0.5% by weight of glycosaminoglycan(s) (for example from 0.5% to 5%) with respect to the dry weight of sucrose (the agar-agar possibly present in the liquid medium will have a content adapted so as to avoid that the culture medium be a substantially complete gel. Advantageously the agar-agar content will be the agar-agar present in the calluse of cells achieved from step 1 or in gel agglomerates containing the plant cells, said calluses or agglomerates being then adapted to move in a liquid phase of the culture medium.);
step 3: the dedifferentiated plant cells are growth in the glycosaminoglycan enriched sucrose liquid culture medium, while being submitted to an elicitation by UV light with a wave length comprised between 200 nm and 300 nm (advantageously between 240 nm and 250 nm), at a temperature comprised between 10° C. and 35° C. for 7 to 30 days, so as to produce encapsulated UV-elicited dedifferentiated plant cells, whereby said encapsulated UV-elicited dedifferentiated plant cells comprise vacuoles comprising at least stilbenes, vitamins, proteins and amino acids, whereby the dry weight of stilbenes selected from the group consisting of resveratrol (especially in the form of trans-resveratrol), catechine (especially as catechin or epicatechin), astringine (especially as trans-astringine and cis-astringine) and piceide (especially as trans-piceide and cis-piceide) with respect to the dry weight of the encapsulated UV-elicited dedifferentiated plant cells before being communited is at least 0.1% (for example comprised between 0.1% and 1%, such as between 0.2% and 0.5%) (the cells can comprise other stilbene compounds), the cell being provided with a bi-lipidic membrane comprising at least C₁₂-C₂₀ fatty acids, cholesterol and ceramides, whereby said encapsulated UV-elicited dedifferentiated plant cells are at least partly encapsulated with glycosaminoglycans;
step 4: the encapsulated UV-elicited dedifferentiated plant cells are recovered from the glycosaminoglycan enriched sucrose liquid culture medium; and
step 5: the encapsulated UV-elicited dedifferentiated plant cells are mixed with cosmetically acceptable excipients at a temperature below 35° C., so as to prepare a cosmetic composition comprising from 0.1% by weight to 5% by weight of said encapsulated UV-elicited dedifferentiated plant cells.

Advantageously, at least one component selected from the group consisting of glycerol, ethyleneglycol, propyleneglycol, diethyleneglycol, butyleneglycol and mixtures thereof is added to the glycosaminoglycan enriched sucrose liquid culture medium, at least at one moment selected from the group consisting of: before the elicitation of the dedifferentiated plant cells by UV light with a wave length comprised between 200 nm and 300 nm, during the elicitation of the dedifferentiated plant cells by UV light with a wave length comprised between 200 nm and 300 nm, after the elicitation of the dedifferentiated plant cells by UV light with a wave length comprised between 200 nm and 300 nm, and combinations thereof, so as to form encapsulated UV-elicited dedifferentiated plant cells encapsulated at least with glycosaminoglycan and at least one component selected from the group consisting of glycerol, ethyleneglycol, propyleneglycol, diethyleneglycol, butyleneglycol and mixtures thereof

According to an embodiment, at least one component selected from the group consisting of glycerol, ethyleneglycol, propyleneglycol, diethyleneglycol, butyleneglycol and mixtures thereof is added to the glycosaminoglycan enriched sucrose liquid culture medium, at least before the elicitation of the dedifferentiated plant cells by UV light with a wave length comprised between 200 nm and 300 nm, so as to form encapsulated UV-elicited dedifferentiated plant cells encapsulated at least with glycosaminoglycan and at least one component selected from the group consisting of glycerol, ethyleneglycol, propyleneglycol, diethyleneglycol, butyleneglycol and mixtures thereof.

According to a detail of an embodiment, in step 2, the dedifferentiated plant cells from the culture of step 1 are put in suspension and mixed in a liquid sucrose containing culture medium enriched with at least 0.5% by weight of glycosaminoglycan selected from the group consisting of Chondroitin and cosmetically acceptable salts thereof, heparin and cosmetically acceptable salts thereof, Heparitin and cosmetically acceptable salts thereof, Hyaluronic Acid and cosmetically acceptable salts thereof, Keratan and cosmetically acceptable salts thereof, and their mixtures, with respect to the weight of sucrose.

According to a further detail of an embodiment, at least for a part of step 3, trehalose is added to the sucrose liquid culture medium, whereby the dedifferentiated plant cells are growth at least partly in a glycosaminoglycan and trehalose enriched sucrose liquid culture medium, while being submitted to an elicitation by UV light with a wave length comprised between 200 nm and 300 nm. Preferably, at least for a latest stage of step 3, trehalose is added to the sucrose liquid culture medium, whereby the dedifferentiated plant cells are growth at least partly in a glycosaminoglycan and trehalose enriched sucrose liquid culture medium, while being submitted to an elicitation by UV light with a wave length comprised between 200 nm and 300 nm.

In said methods of the invention, one or more parameters are adapted so as to achieve one or more characteristics of a composition of the invention.

The invention further relates to a method of preparing a cosmetic composition for topical application containing at least:

(a) a powder of communited lyophilized encapsulated UV-elicited dedifferentiated plant cells, whereby said encapsulated UV-elicited dedifferentiated plant cells comprise before being communited vacuoles comprising at least stilbenes, vitamins, proteins and amino acids, whereby the dry weight of stilbenes selected from the group consisting of resveratrol (especially in the form of trans-resveratrol), catechine (especially as catechin or epicatechin), astringine (especially as trans-astringine and cis-astringine) and piceide (especially as trans-piceide and cis-piceide) with respect to the dry weight of the encapsulated UV-elicited dedifferentiated plant cells before being communited is at least 0.1% (for example comprised between 0.1% and 1%, such as between 0.2% and 0.5%) (the cells can comprise other stilbene compounds), the encapsulated UV-elicited dedifferentiated plant cells before being communited being provided with a bi-lipidic membrane comprising at least C₁₂-C₂₀ fatty acids, cholesterol and ceramides, whereby said encapsulated UV-elicited dedifferentiated plant cells before being communited are at least partly encapsulated with glycosaminoglycans, whereby the cosmetic composition comprises from 0.1% by weight to 5% by weight of said powder of communited lyophilized encapsulated UV-elicited dedifferentiated plant cells, and

(b) a cosmetically acceptable excipient, said method comprising at least the following steps:

step 1: dedifferentiated plant cells are growth in vitro in an agar-agar containing culture medium in a sterile atmosphere at a temperature comprised between 10° C. and 35° C., so as to form a culture of dedifferentiated plant cells;
step 2: dedifferentiated plant cells from the culture of step 1 are put in suspension and mixed in a liquid sucrose containing culture medium enriched with at least 0.5% by weight of glycosaminoglycan with respect to the dry weight of sucrose (the agar-agar content will be adapted so as to avoid that the culture medium be a substantially complete gel. Advantageously the agar-agar content will be adapted so as to gel agglomerates containing the plant cells adapted to move in a liquid phase of the culture medium.);
step 3 the dedifferentiated plant cells are growth in the glycosaminoglycan enriched sucrose liquid culture medium, while being submitted to an elicitation by UV light with a wave length comprised between 200 nm and 300 nm (advantageously between 240 nm and 280 nm), at a temperature comprised between 0° C. and 35° C. for 7 to 30 days, so as to produce encapsulated UV-elicited dedifferentiated plant cells, whereby said encapsulated UV-elicited dedifferentiated plant cells comprise vacuoles comprising at least stilbenes, vitamins, proteins and amino acids, whereby the dry weight of stilbenes selected from the group consisting of resveratrol (especially in the form of trans-resveratrol), catechine (especially as catechin or epicatechin), astringine (especially as trans-astringine and cis-astringine) and piceide (especially as trans-piceide and cis-piceide) with respect to the dry weight of the encapsulated UV-elicited dedifferentiated plant cells before being communited is at least 0.1% (for example comprised between 0.1% and 1%, such as between 0.2% and 0.5%) (the cells can comprise other 1 is stilbene compounds), the cell being provided with a bi-lipidic membrane comprising at least C₁₂-C₂₀ fatty acids, cholesterol and ceramides, whereby said encapsulated IV-elicited dedifferentiated plant cells are at least partly encapsulated with glycosaminoglycans;
step 4: the encapsulated UV-elicited dedifferentiated plant cells are recovered from the glycosaminoglycan enriched sucrose liquid culture medium;
step 5 the recovered encapsulated UV-elicited dedifferentiated plant cells are lyophilised and communited in a powder, and
step 6: powder of comminuted lyophilised UV-elicited dedifferentiated plant cells is mixed with cosmetically acceptable excipients at a temperature below 35° C., so as to prepare a cosmetic composition comprising from 0.1% by weight to 5% by weight of said powder of comminuted lyophilised UV-elicited dedifferentiated plant cells.

Advantageously in said method, at least one component selected from the group consisting of glycerol, ethyleneglycol, propyleneglycol, diethyleneglycol, butyleneglycol and mixtures thereof is added to the glycosaminoglycan enriched sucrose liquid culture medium, at least at one moment selected from the group consisting of: before the elicitation of the dedifferentiated plant cells by UV light with a wave length comprised between 200 nm and 300 nm, during the elicitation of the dedifferentiated plant cells by UV light with a wave length comprised between 200 nm and 300 nm, after the elicitation of the dedifferentiated plant cells by UV light with a wave length comprised between 200 nm and 300 nm, and combinations thereof, so as to form encapsulated UV-elicited dedifferentiated plant cells encapsulated at least with glycosaminoglycan and at least one component selected from the group consisting of glycerol, ethyleneglycol, propyleneglycol, diethyleneglycol, butyleneglycol and mixtures thereof.

According to a detail of an embodiment, at least one component selected from the group consisting of glycerol ethyleneglycol, propyleneglycol, diethyleneglycol, butyleneglycol and mixtures thereof is added to the glycosaminoglycan enriched sucrose liquid culture medium, at least before the elicitation of the dedifferentiated plant cells by UV light with a wave length comprised between 200 nm and 300 nm, so as to form encapsulated UV-elicited dedifferentiated plant cells encapsulated at least with glycosaminoglycan and at least one component selected from the group consisting of glycerol, ethyleneglycol, propyleneglycol, diethyleneglycol, butyleneglycol and mixtures thereof.

Advantageously, in step 2, the dedifferentiated plant cells from the culture of step 1 are put in suspension and mixed in a liquid sucrose containing culture medium enriched with at least 0.5% by weight of glycosaminoglycan selected from the group consisting of Chondroitin and cosmetically acceptable salts thereof, heparin and cosmetically acceptable salts thereof, Heparitin and cosmetically acceptable salts thereof, Hyaluronic Acid and cosmetically acceptable salts thereof, Keratan and cosmetically acceptable salts thereof, and their mixtures, with respect to the weight of sucrose.

Preferably, at least for a part of step 3, trehalose is added to the sucrose liquid culture medium, whereby the dedifferentiated plant cells are growth at least partly in a glycosaminoglycan and trehalose enriched sucrose liquid culture medium, while being submitted to an elicitation by UV light with a wave length comprised between 200 nm and 300 nm.

Most preferably, at least for a latest stage of step 3, trehalose is added to the sucrose liquid culture medium, whereby the dedifferentiated plant cells are growth at least partly in a glycosaminoglycan and trehalose enriched sucrose liquid culture medium, while being submitted to an elicitation by UV light with a wave length comprised between 200 nm and 300 nm.

In said methods of the invention, one or more parameters are adapted so as to achieve one or more characteristics of a composition of the invention.

The composition of the invention comprises all or substantially all the substances present in the encapsulated UV-elicited dedifferentiated plant cells, among others cell membranes, cytoplasmic organisms and vacuole material. The integrity of the elicited plant cells can thus be maintained, even for a long period.

The plant cells present in the composition of the invention have advantageously the advantage of deactivating oxidising enzymes without additions of additives or chemical products, by the encapsulation techniques, possibly followed by drying operation, such as a freeze drying.

The plant cells present in the compositions of the invention have a higher content in phytoalexins, preferably in stilbenes, with respect to the content present in fresh plant cells.

The expression “elicitation in the culture medium” should be understood to mean subjecting the cells to UV—stress or attack in their culture medium in order to trigger one or more defence mechanisms, said stress being possibly combined with one or more other stresses such as biological, chemical or physical.

During these UV stresses, three categories of defence systems can be activated:

the formation of a healing epidermis and reinforcement of the walls (lignification, etc.) (Dai et al. 1995);

the synthesis of defence proteins or “Pathogenesis Related” (PR) proteins discovered in 1970 by the tobacco industry. These PRs, for example, include protease inhibitors (Ryan, 1992), hydrolytic enzymes such as chitinases or β-1,3-glucanases (Derckel et al., 1996; Robinson et al., 1997, Kraevas et al., 1998; Salzman et al., 1998; Renault et al., 2000);

and the synthesis of secondary metabolites of the phytoalexin type. Of these secondary metabolites, more than 300 phytoalexins have already been characterised. They form part of a large spectrum of different chemical classes which include coumarins, benzofurans, terpenes, alkaloids, certain polyphenols (Smith, 1996), etc.

The implementation of defence reactions of plants involves a whole panoply of transduction signals which result in the rapid induction of the expression of defence genes. Thus, the recognition of the pathogen by the host plant activates a cascade of signals in the attacked cells, such as the phosphorylation of proteins by protein kinases, the flow of ionic species (Ca²⁺), the formation of reactive oxygenated species (Coté and Hahn, 1994; Shibuya et al., 1996; Benhamou, 1996), etc.

Moreover, the attacked cells are capable of producing alarm signals which are transmitted to adjacent cells (local reaction) as well as to the whole plant, and which thus generate the systemic reaction phenomenon, as stated in the previous paragraph.

The most studied mechanism of systemic resistance is the phenomenon of SAR or “systemic acquired resistance.” The term SAR was defined by Ross in 1961. It describes the appearance of the resistance of a plant following an attack by a pathogen, both in the infected parts and in the healthy parts of the plant. In general, it is developed after the appearance of necrotic lesions around the inoculation site. This localised hypersensitivity response restricts the pathogen to within and around the site of infection, and appears to make the plant more resistant to attack by various organisms (Ryals et al., 1996). How are plant parts remote from the infection site capable of acquiring this resistance? In 1966 Ross developed the idea of the existence of signal molecules which, at low concentrations, are capable of activating defence mechanisms in tissues remote from the infected zone.

In the vine, the signalling mechanisms involved in the expression of defence reactions are still not well known. However, the synthesis occurs of three types of defence molecules (lignin, defence proteins and phytoalexins). In particular, the role of phytoalexins is played by a family of original compounds, namely polyphenols (Deloire et al., 2000).

Present in more or less large amounts in all the organs of the plant, phytoalexins can be induced in leaves and berries. This type of induction is designated by the term “elicitation”. Elicitation factors (or elicitors) can have different origins. Elicitation can take the form of:

biotic elicitation, for example on an attack by a pathogen such as Botrytis cinerea, a grey rot agent (Jeandet et al., 1995; Bavaresco et al., 1997), Plasmopara viticola, a mildew agent (Dereks and Creasy, 1989) or Phomopsis viticola, which is responsible for exconrosis (Hoos and Blaich, 1990).

abiotic elicitation by environmental factors such as UV, temperature, light, asphyxia, natural agents extracted from other plants (Jeandet et al., 1997; Langcake and Pryce, 1977b; Douillet-Breuil et al., 1999), aluminium chloride (Adrian et al., 1996) or ozone (Sarig et al., 1996).

On elicitation, phytoalexins such as trans-resveratrol, trans-piceid, e-viniferin and pterostilbene can be induced in leaves and berries (Soleas et al., 1997). This property of the de novo biosynthesis of phytoalexins in response to a stress, particularly after attack by a pathogen, suggests that these molecules could play the role of natural means of defence of the plants.

This role of defence molecules is corroborated by certain studies which seem to indicate a close interrelationship between the level of natural resistance of the plant and its ability to synthesise these molecules. For example, Langcake and McCarthy (1979) demonstrated a relation between the resistance of certain species of the Vitis kind to Botrytis cinerea or Plasmopara viticola and their capacity for the biosynthesis of phytoalexins (resveratrol and viniferin). Moreover, Dercks and Creasy (1989) showed that species resistant to Plasmopara viticola produce five times more phytoalexins than do sensitive species. Similarly, within the Vinifera species there are some vines which are more or less tolerant to attack by fungi depending on their capacity for producing phytoalexins.

According one preferred embodiment, the dedifferentiated cells are UV-elicited in an in vitro culture medium under a CO₂ rich atmosphere, i.e. an atmosphere containing more than 5%, advantageously more than 10%, preferably more than 20% by volume CO₂. The temperature of the culture medium during the elicitation is advantageously comprised between 20 and 35° C.

The elicitation step is advantageously controlled so that a specific brown colour or light brown colour is achieved. When said brown or light brown colour is achieved, the elicitation is stopped or continued for still a further period of at least 12 hours, preferably at least about one day. When the brown or light brown colour is obtained, UV-elicited dedifferentiated plant cells can be chemically analysed, for example via HPLC, in order to control the content in phytoalexins of the elicited plant cells.

Advantageously, during the growing step of dedifferentiated plant cells in the in vitro culture medium, some elicited plant cells are added.

According to one particular embodiment, the dedifferentiated plant cells are subjected successively to several in vitro culture stages without elicitation and to one or a plurality of in vitro culture stages with elicitation.

The encapsulated UV-elicited dedifferentiated plant cells are advantageously rich in flavonoids such as flavanols, anthocyans and flavonols, with respect to fresh plant cells.

The encapsulated UV-elicited dedifferentiated plant cells when pressed on the skin liberate one or more metabolites having one or more one or more activities selected from antioxidant, anti-radical, anti-inflammatory, anti-proliferative, relaxant and vascular activities, etc. The pressure exerted on the skin during a massage breaks the thin layer encapsulating the UV elicited dedifferentiated plant cells, whereby releasing metabolites inside the plant cells to the skin.

The expression “composition for topical use” should be understood to mean creams, ointments, lotions, suspensions, sticks, shampoos, gels, solutions (applicable by spraying, for example).

The present invention further relates to a method for the cosmetic treatment of skin, characterised in that a composition according to the invention is applied to the skin, hair, and/or mucous membranes.

In particular, the cosmetic treatment method of the invention can be put into effect by applying the cosmetic compositions as defined above by the customary technique for the use of these compositions. For example: the application of creams, gels, serums, lotions, milks, shampoos or sun-reflective compositions to the skin.

In these examples, preferred methods for the preparation of cosmetic compositions are disclosed hereafter.

In said description, reference is made to the following drawings.

### BRIEF DESCRIPTION OF THE DRAWINGS

FIG. 1 is a schematic chart of a first method of the invention;

FIG. 2 is a schematic chart of a second method of the invention

FIGS. 3 to 5 are the HPLC curves (by fluorescence), respectively of the Fresh vine cells of green grape (cabernet sauvignon), dedifferentiated vine cells (in vitro culture as for example 1C, but without UV-elicitation), and Encapsulated UV-elicited dedifferentiated vine cells (example 1C);

FIGS. 6 to 8 are the HPLC curves (by UV absorption at 286 nm), respectively of the Fresh vine cells of green grape (cabernet sauvignon), dedifferentiated vine cells (in vitro culture as for example 1C, but without UV-elicitation), and Encapsulated UV-elicited dedifferentiated vine cells (example 1C).

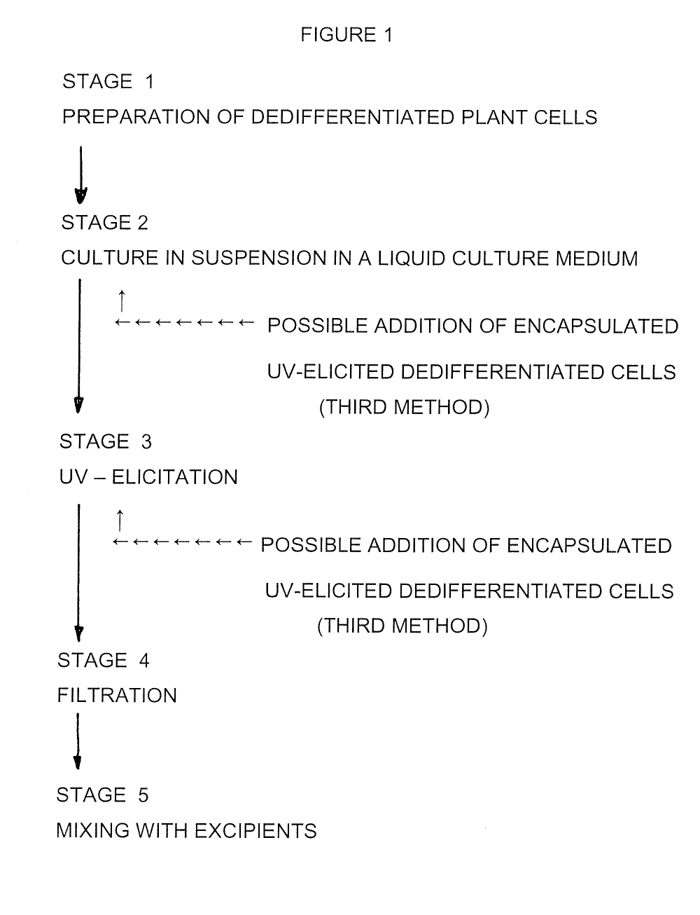

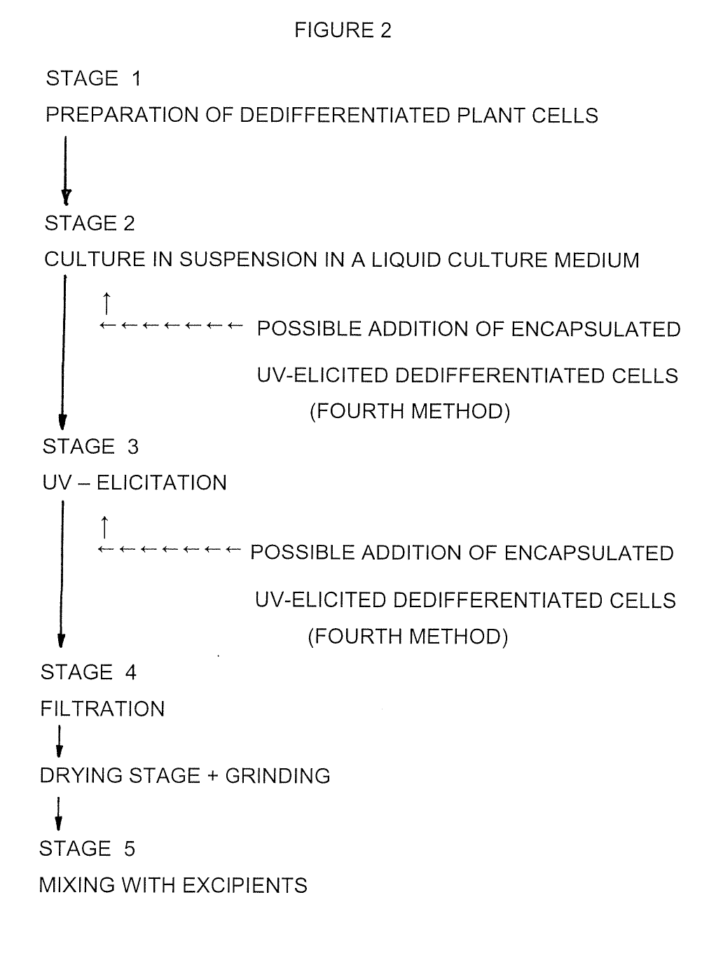

### DESCRIPTION OF PREFERRED METHODS OF THE INVENTION

### First Method

### Stage 1: Preparation of Dedifferentiated Plant Cells

Preparation of Dedifferentiated Cells Cultivated in an In Vitro Culture Medium. Said step is operated in vitro on an agar-agar containing culture medium. The culture medium comprise saccharose, vitamins and other salts, as required for the growth of plant cells. This operation is carried out in a sterile atmosphere at a temperature comprised between 20° C. and 30° C. The preferred plant cells is vine cells (vitis), most preferably Cabernet sauvignon vine cells.

The first step for the development of plant cell cultures consists of selecting the plant which produces the sought-after substances. It is nowadays acknowledged that within the same species there is a variability of the production capacities for a given metabolite, part of which variability is of genetic origin. When it is possible, it is therefore necessary to exploit this variability by selecting the best genotype, i.e. the one which is the most productive for the sought-after metabolite. Primary proliferations can successfully be induced from sterilised fragments of a selected plant organ (leaf, stem, root, etc.), placed in vitro on a solid medium (gelose/agar-agar). Thus, after some weeks in culture, undifferentiated accumulations of cells termed calluses are formed in the explants. The growth of these calluses is maintained by successive subculturing stages on a new nourishing medium. These conditions of culture induce the spontaneous appearance of morphological and metabolic-variability between calluses derived from the same plant or the same explant.

The maintenance of constant environmental conditions when subculturing the plant cells tends to reduce this variability of the plant cells. Thus, after one to two years of regular subculturing, a collection of stable strains is obtained which exhibit the growth and production characteristics of very different metabolites. At this stage, it is then possible, with the help of tests, to select the strain or strains which produce a significant amount of the compounds of interest. Introducing these calluses into a liquid environment then enables progress to be made move towards larger production volumes, firstly in 250 ml phials, and subsequently in a bio-reactor (20 litres or more).

Example of solid culture medium used is:

For one liter of aqueous growth solid (gel) culture medium

| | | |
|---|---|---|
| KH₂PO₄•H₂O | 276 | mg |
| Ca₃(PO₄)₂ | 200 | mg |
| (NH₄)₂SO₄ | 500 | mg |
| MgSO₄•7H₂O | 122 | mg |
| KNO₃ | 525 | mg |
| FeSO₄•7H₂O | 14 | mg |
| Na₂EDTA•2H₂O | 19 | mg |
| MnSO4•H2O | 6 | mg |
| H₃BO₃ | 1.5 | mg |
| ZnSO₄•7H₂O | 2.7 | mg |
| CuSO₄•5H₂O | 0.05 | mg |
| Nicotinic acid | 0.5 | mg |
| Thiamine hydrochloride | 2 | mg |
| Pyridoxal hydrochloride | 0.25 | mg |
| Biotin | 0.25 | mg |
| Glycin | 2 | mg |
| myo-Inositol | 25 | mg |
| 6-dimethylallylamino purine | 3 | mg |
| 1-Naphtaylacetic acid | 0.001 | mg |
| Sucrose | 20000 | mg |
| Casein Enzymatic Hydrolysate | | |
| NZ-Amine A | 2000 | mg |
| Agar (gelose) | 8000 | mg |
| Jasminate (methyl) | 20 to 100 | mg |
| Glycosaminoglycans (such as heparin) | 100 to 500 | mg |

The pH of the medium was to 5.6, and the medium was sterilised at 121° C. for at least 20 minutes, for example for 20 to 60 minutes.

### Stage 2: Culture in Suspension in a Liquid Culture Medium

A liquid culture medium is prepared, said medium comprising some saccharose, as well as some vitamins. To said liquid medium, glycosaminoglycans are added so that the concentration in glycosaminoglycans in the liquid culture medium is comprised between 1 and 5% by weight.

Agglomerates of plant cells attached to agar-agar gel particles or callus of cells are prepared and added slowly in the liquid medium under slight mixing. In some other example, plant cells recovered from stage 1 were mixed to the liquid culture medium free or substantially free from agar.

The temperature of the medium is maintained at about 20-30° C., while the atmosphere is sterile.

For said growth of dedifferentiated plant cells, the liquid culture medium is placed in vials provided with a cap, whereby the gas atmosphere within the vial can be enriched with CO₂. (CO₂ volume content of 1 to 10%)

The plant cells in suspensions are formed from aggregates and isolated cells. In case of heterogeneity (somaclonal variability), an additional selection can be made in order to obtain highly productive cell lines. In addition to this cloning operation, the production of the metabolite of interest can also be optimised by modifying the culture conditions, resulting in the development of media termed production media. This liquid medium is identical to the cell subculture medium except for the concentration of sucrose, which is multiplied by two. During their culture in a production medium, highly productive, and except that no agar-agar is added or that the amount of agar-agar is not sufficient for achieving a complete gel, but that gel agglomerates are floating on or flowing in the liquid culture medium.

Advantageously some unsaturated fatty acids and/or pectin are added to the liquid culture medium. Also some vegetable oil, such as olive oil, is advantageously added to the liquid culture medium, so as to form a liquid barrier floating on the liquid culture medium.

Glycerol and/or butylenes glycol is/are also advantageously added to the liquid culture medium.

Example of liquid culture medium is given herebelow:

| | | |
|---|---|---|
| KH₂PO₄•H₂O | 276 | mg |
| Ca₃(PO₄)₂ | 200 | mg |
| (NH₄)₂SO₄ | 500 | mg |
| MgSO₄•7H₂O | 122 | mg |
| KNO₃ | 525 | mg |
| FeSO₄•7H₂O | 14 | mg |
| Na₂EDTA•2H₂O | 19 | mg |
| MnSO4•H2O | 6 | mg |
| H₃BO₃ | 1.5 | mg |
| ZnSO₄•7H₂O | 2.7 | mg |
| CuSO₄•5H₂O | 0.05 | mg |
| Nicotinic acid | 0.5 | mg |
| Thiamine hydrochloride | 2 | mg |
| Pyridoxal hydrochloride | 0.25 | mg |
| Biotin | 0.25 | mg |
| Glycin | 2 | mg |
| myo-Inositol | 25 | mg |
| 6-dimethylallylamino purine | 3 | mg |
| 1-Naphtaylacetic acid | 0.001 | mg |
| Sucrose | 20000 | mg |
| Casein Enzymatic Hydrolysate | | |
| NZ-Amine A | 2000 | mg |
| Jasminate (methyl) | 20 to 100 | mg |
| olive oil | 0 to 100 | mg |
| Glycosaminoglycans (such as heparin) | 100 to 500 | mg |
| glycerol or butylene glycol | 25 to 200 | mg |
| gelose | 0 to less than 100 | mg |

The pH of the medium was to 5.6, and the medium was sterilised at 121° C. for at least 20 minutes, for example for 20 to 60 minutes.

### Stage 3: UV-Elicitation

The dedifferentiated plant cells are further growth in the liquid culture medium, advantageously after adapting the glycosaminoglycans content in the liquid phase between 1 and 5% by weight. Glycerol and/or butylene glycol, unsaturated fatty acid and/or pectin are added to the liquid culture medium.

The plant cells, advantageously the vine cells, most preferably the Cabernet Sauvignon vine cell lines are elicited, after their inoculation of culture in a liquid culture medium for at least 7 days, for example from 8 to 15 days, preferably at least about ten days after their inoculation, by 254 nm UV light from a Wilber-Lourmat T-30C lamp (600 μW/m2) placed at a distance of 1 m to provide direct illumination of the cells for successive periods of 10 minutes UV light, with intermediate period with no UV light for period of 10 minutes to 2 hours, preferably about 1 hour (during said intermediate periods, advantageously the culture medium are in placed in a dark atmosphere), which induces a considerable accumulation of polyphenols, particularly stilbenes, as well as other interesting active components in the cells. This means of elicitation clearly does not form any impurity in the cell culture. The elicitation step is carried for 7 to 30 days, advantageously about 10 days.

The end of the elicitation stage period, before trehalose is added to the liquid culture medium, is advantageously controlled by determining the colour of the UV-elicited dedifferentiated plant cells. When the colour (brown, beige) is substantially stable, the UV-elicitation step can be further carried out with trehalose for a short period.

For the latest elicitation days or day, trehalose is added to the culture medium. Trehalose is for example added to the liquid medium at the rate of 100 mg to 1000 mg/liter.

At the end of the elicitation step, HPLC analysis is advantageously carried out as quality control so as to confirm that the dry weight of stilbenes selected from the group consisting of resveratrol (especially in the form of trans-resveratrol), catechine (especially as catechin or epicatechin), astringine (especially as trans-astringine and cis-astringine) and piceide (especially as trans-piceide and cis-piceide) with respect to the dry weight of the encapsulated UV-elicited dedifferentiated plant cells before being communited is at least 0.1% (for example comprised between 0.1% and 1%, such as between 0.2% and 0.5%) (the encapsulated UV-elicited dedifferentiated plant cells can comprise other stilbene compounds).

### Stage 4: Filtration

At the end of the elicitation stage, the plant cells are filtered to remove the remaining liquid culture medium and are rinsed in cold water (4° C.).

A fresh biomass of about 300 to 500 grams per litre of encapsulated UV-elicited plant cells is thus obtained.

Possibly the water content of said fresh biomass can be controlled, and some more glycerol or butylenes glycol can be further added to the fresh biomass, so as to form a suspension or a gel, ready to be mixed with cosmetic excipients.

Stage 5: Mixing with Excipients

The excipients will be cosmetically acceptable excipients used in a concentration not sufficient to damage or to destroy the protective coating layer of the plant cells.

The mixing operation will be carried out under sterile atmosphere at a temperature lower than 35° C. The mixer used for said mixing will also be selected so as ensure a gentle mixing, not destroying or damaging the coating layer encapsulating the UV-elicited plant cells.

The cosmetic composition comprises advantageously from 0.5 to 1.5% by weight of encapsulated UV-elicited dedifferentiated plant cells.

### Second Method

Stages 1 to 4 of the first method are repeated.

After the filtration stage, the UV-elicited dedifferentiated plant cells are submitted to a drying step, such as a freeze drying stage, so that the content of the UV-elicited dedifferentiated encapsulated plant cells is reduced to 3 to 10% by weight. The so dried plant cells are then communited in a gentle manner so as not to destroy or damage completely the coating layer covering the plant cells.

About 20 to 50 grams of dry biomass (encapsulated plant cells) per litre of culture are obtained after freeze-drying in a Virtis apparatus (Uni-Trap 10-100).

The drying operation is conducted so as not to destroy the structure of the plant cell membranes. This drying is advantageously conducted at a temperature less than 35° C., for example between −60° C. and 0° C.

After said communition step, a powder of plant cells is obtained, the particles of said powder having an average particle size of about 1 μm.

It should also be noted that communition of the dedifferentiated, UV-elicited encapsulated cells is advantageously effected in the presence of one or more agents or excipients of the cosmetic composition.

The comminuted product thus comprises encapsulated plant cells containing substantially all the dry components which form the cell, i.e. substantially all the components of the membrane, of the cytoplasm and of the vacuoles. Said powder is then mixed with cosmetically acceptable excipients, as in stage 5 of the first method.

The cosmetic composition comprises advantageously from 0.5 to 1.5% by weight of encapsulated UV-elicited dedifferentiated plant cells.

### Third and Fourth Methods

These methods are similar to the first and second methods, except that previously prepared encapsulated UV-elicited dedifferentiated plant cells are added to the liquid culture medium before/during the elicitation stage.

### Examples of Encapsulated UV-Elicited Dedifferentiated Plant Cells and Their Uses

### Example 1

Encapsulated UV-elicited dedifferentiated plant cells were prepared from various materials. The data are summarised in the following Table:

| | Material from which the | |
|---|---|---|
| Example 1 (vine) | dedifferentiated cells | |
| Cabernet sauvignon | originate | Type of elicitation |
| 1A | branch less than one year | UV radiation for 20 |
| | old | days |
| 1B | cuticle of ripe grape | UV radiation for 24 |
| | | days |
| 1C | cuticle of green grape | UV radiation for 24 |
| | | days |
| 1D | grape seed | UV radiation and |
| | | carbon dioxide for 20 |
| | | days |
| 1E | root | UV radiation for 25 |
| | | days |
| 1F | green leaf | UV radiation for 25 |
| | | days |
| 1G | bud | UV radiation for 20 |
| | | days |
| 1H | residue from a pressing | UV radiation for 20 |
| | stage | days |
| 1I | residue from a pressing | UV radiation for 30 |
| | stage | days |

The stilbene content of the UV-elicited encapsulated dedifferentiated plant cells has been determined by HPLC.

### Materials and Methods:

Bischoff Model 2,200 pump

automatic injector (Alcoot Model 788 autosampler)

Ultrasep C18 column (30×cm 0.18 cm); porosity 6 mm

Jasco 821-FI fluorescence detector.

Fluorescence was detected with excitation at 280 nm and emission at 310 nm and/or the detection was carried out by UV absorption at 286 nm. The eluant used was composed of methanol: water, 40:60 (v/v), the pH of which was adjusted to 8.3 with 1M KOH.

The following plant cells have been analysed:

Encapsulated UV-elicited dedifferentiated vine cells (example 1C) (vine cells of the invention)

dedifferentiated vine cells (in vitro culture as for example 1C, but without UV-elicitation) (non UV elicited dedifferentiated vine cells)

Fresh plant cells from the cuticle of green grape (cabernet Sauvignon) (fresh vine cells)

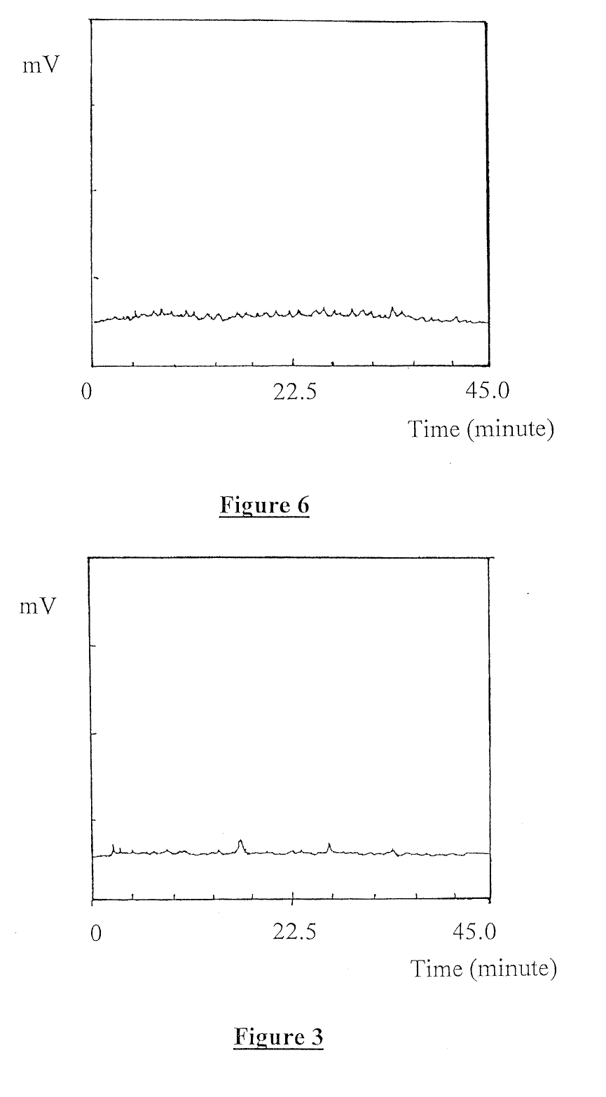

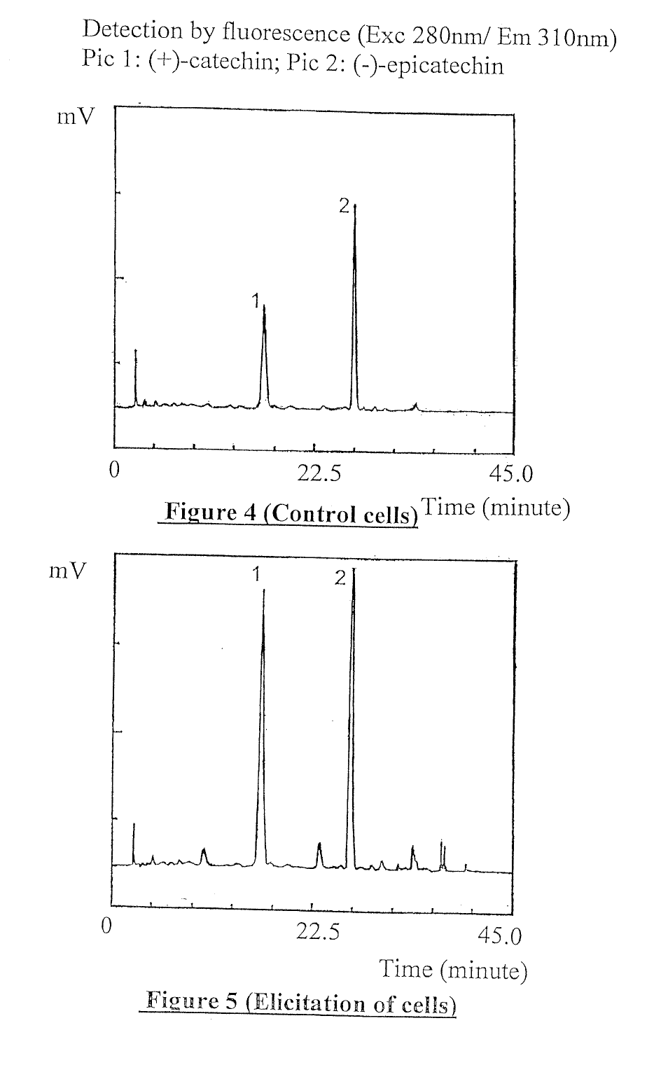

FIGS. 3 to 5 are the HPLC curves (by fluoresence), respectively of the Fresh vine cells of green grape (cabernet sauvignon), dedifferentiated vine cells (in vitro culture as for example 1C, but without UV-elicitation), and Encapsulated UV-elicited dedifferentiated vine cells (example 1C). The stilbene content of the fresh vine cells from green grape was substantially nihil.

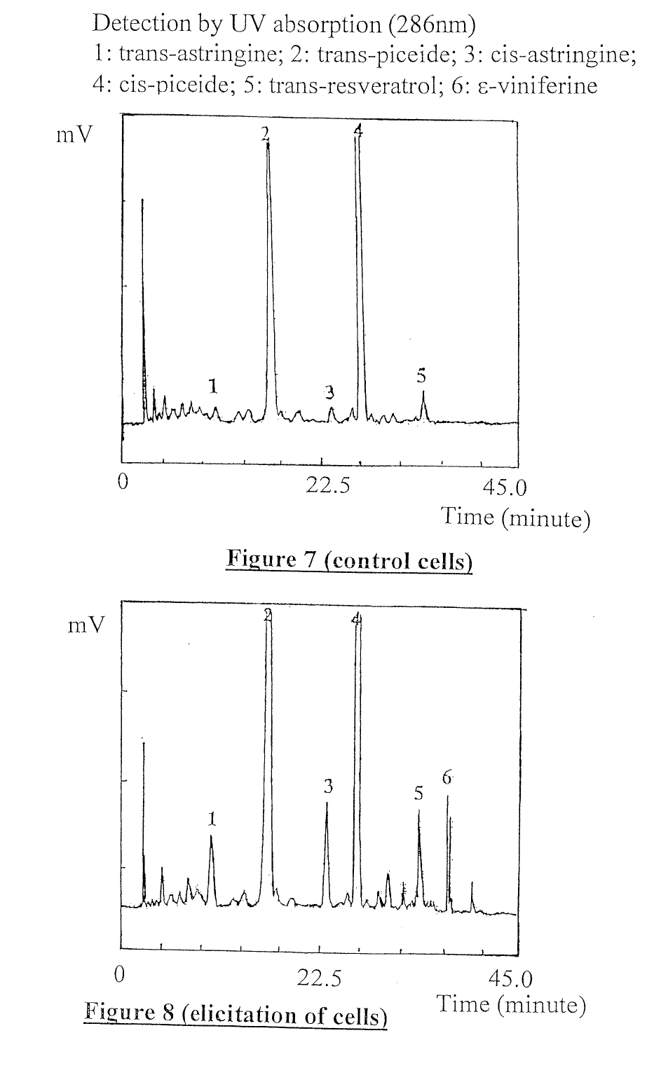

FIGS. 6 to 8 are the HPLC curves (by UV absorption at 286 nm), respectively of the Fresh vine cells of green grape (cabernet sauvignon), dedifferentiated vine cells (in vitro culture, as for example 1C, but without UV-elicitation), and Encapsulated UV-elicited dedifferentiated vine cells (example 1C). The stilbene content of the fresh vine cells from green grape was substantially nihil.

The following table gives the content of some stilbene compounds expressed in nano moles/gram of dry weight of the different vine cells.

| | | | encapsulated |
|---|---|---|---|
| | | dedifferentiated | dedifferentiated |
| stilbene | | cells non UV | UV-elicited plant |
| compound | | elicited | cells |
| | Fresh cells | | |
| catechine | less than 10 | 225 | more than 500, |
| | | | such as between |
| | | | 500 and 1000, for |
| | | | example about |
| | | | 550 |
| | fresh cells | | |
| Epicatechine | less than 10 | 314 | more than 500, |
| | | | such as between |
| | | | 500 and 1000, for |
| | | | example about |
| | | | 600 |
| Trans-astringine | less than 10 | 25 | more than 100, |
| | | | such as between |
| | | | 100 and 500, for |
| | | | example about |
| | | | 150 |
| Cis-astringine | less than 10 | 42 | more than 100, |
| | | | such as between |
| | | | 100 and 500, for |
| | | | example about |
| | | | 200 |
| Trans-piceide | less than 10 | 280 | more than 300, |
| | | | such as between |
| | | | 300 and 600, for |
| | | | example about |
| | | | 350 |
| Cis-piceide | less than 10 | 230 | more than 300, |
| | | | such as between |
| | | | 300 and 600, for |
| | | | example about |
| | | | 320 |
| Trans-resveratrol | less than 10 | 325 | more than about |
| | | | 600, such as |
| | | | between 600 and |
| | | | 2000, for example |
| | | | from about 900 to |
| | | | 1500 |

As further comparative step, dedifferentiated UV-elicited cabernet sauvignon cells have been prepared by a method similar to the first method, except that no glycosaminoglycans and trehalose were added to the culture medium. The so achieved dedifferentiated UV-elicited plant cells were not stable and had a variable stilbene content. After less than 1 week a drop in stilbene content was observable to about the stilbene content of dedifferentiated cell without UV-elicitation. A rapid colouration and odour happened.

From said analysis it appears that the encapsulated UV-elicited cabernet sauvignon cells are rich in stilbene compounds, such as resveratrol, piceide, astringine and catechine. The colour of the cells was beige brown. No specific odour was emitted from the cells.

When carrying back said analysis, 3 months, 6 months and 12 months after the preparation of the encapsulated UV-elicited cabernet sauvignon cells of the invention, it appears that the stilbene content was substantially not modified, while no major difference in colour and odour was found. The encapsulated cells of the invention are thus stable.

### Example 2

### Pharmacological Activity of the Encapsulated UV-Elicited Dedifferentiated Cabernet Sauvignon Vine Cells

The anti-radical and anti-oxidant activity of the product obtained according to Example 1A was investigated in vitro. A SKINETHIC® reconstituted model epidermis was used, which enabled this activity to be revealed by the determination of malondialdehyde (MDA) after the induction thereof by ultraviolet B radiation.

For said test, plant cells prepared according to the first method or according to the second method have been used. Said cells were prepared one year before the test, so as to confirm the stability of the encapsulated UV-elicited dedifferentiated plant cells of the invention.

Said tests have shown that the encapsulated UV-elicited dedifferentiated plant cell of the invention had an antioxidant activity and an anti-radical effect just after preparation, as well as 3) months, 6 months and 12 months after their preparation.

### Example 3

### Dispersion of Elicited Whole Vine cells in a Cosmetic Base

Vine cells were obtained as described in Examples 1A to 1I. The cells of these examples were used separately or in admixture for the preparation of a cosmetic composition. The cells as a suspension or gel (first method) or as freeze dried particles (second method) were dispersed in the following base:

| | |
|---|---|
| deionised water | 85.31% |
| mineral oil | 9.00% |
| cetyl alcohol | 3.00% |
| ceteareth-20 | 0.75% |
| encapsulated vine cells | 0.50% |
| fragrance | 0.15% |
| carbomer | 0.10% |
| methylchloroisothiazoline | 0.065% |
| and methylisothiazoline [kathon CG] | |
| sodium hydroxide (45%) | 0.06% |
| butylated hydroxyanisole | 0.06% |
| TOTAL | 100.00% |

The composition obtained exhibited a homogeneous dispersion of the cells in the cream and a very fine particle size. A test for cleanliness showed the absence of germs and fungi as well as a remarkable stability of the composition for more than one year. The result obtained from a transcutaneous investigation showed the passage of the active constituents, particularly polyphenols, through cutaneous tissue.

It was observed that when applying the composition on the human skin with pressure, the encapsulated UV-elicited dedifferentiated plant cells contacting the skin entered into the stratum corneum, while the outer capsule and membrane of the plant cells were cut and were cut, whereby enabling the release of the active ingredient present within the cells into the stratum corneum.

As the active agents within the cells are only liberated when exerting pressures it is possible to liberate the active agents only at the places of the skin where required.

### Example 4

### Dispersion of Elicited Whole Vine Cells in a Cosmetic Base

Vine cells were obtained as described in Examples 1A to 1I. The cells of these examples were used separately or in admixture for the preparation of a cosmetic composition. The cells as prepared in accordance to the first method (as a suspension in glycerol) or as a dry powder (second method) were dispersed, in the following base:

| | |
|---|---|
| water | 46.09% |
| sodium lauryl sulphate (25%) | 36.40% |
| PEG-7 glyceryl cocoate | 2.00% |
| laureth-2 | 1.50% |
| laureth-11 sodium carboxylate | 4.00% |
| cocamidopropyl betaine & benzoic acid | 3.48% |
| sodium chloride | 1.60% |
| propylene glycol | 1.00% |
| fragrance | 0.13% |
| PEG-40 hydrogenated castor oil | 0.50% |
| & propylene glycol & water | |
| oleth-10 | 0.50% |
| sodium phosphate | 0.30% |
| disodium phosphate | 0.08% |
| citric acid (50%) | 0.52% |
| sodium benzoate | 0.50% |
| vine cells | 0.50% |
| glycerol | 0.50% |
| salicylic acid | 0.20% |
| phenoxyethanol | 0.20% |
| TOTAL | 100.00% |

The composition obtained exhibited a homogeneous dispersion of cells in the cream and a very fine particle size. A test for cleanliness showed the absence of germs and fungi as well as a remarkable stability of the composition. The result obtained from a transcutaneous investigation showed the passage of the active constituents, particularly polyphenols, through cutaneous tissue.

### Example 5

### Creams

aqueous phase A: demineralised water combined with a moisturising product

oleaginous phase B: emulsifier+emollient+oil

phase C; preservative, perfume

phase D: active substance: comminuted product of dedifferentiated, elicited vine cells, as a viscous suspension or a gel or a substantially dry powder.

### Example 6

### Lotions

Containing an aqueous phase A only: demineralised water, propylene glycol, preservative, perfume, butylene glycol or glycerol and active substance: comminuted product of encapsulated dedifferentiated, elicited vine cells, as a viscous suspension or a gel or a substantially dry powder.

### Example 7

### Shampoos

Containing an aqueous phase A only, based on demineralised water, detergents, foaming agents, thickeners, perfume and active substance: comminuted product of dedifferentiated, elicited vine cells, as a comminuted product of encapsulated dedifferentiated, elicited vine cells, as a viscous suspension or a gel or a substantially dry powder.

### Example 8

### Gels

Hydrogels and oleogels, obtained by the addition of emulsifiers and thickeners to the aqueous phase A or to the oleaginous phase E

phase C: perfume, preservative

phase D: comminuted product of dedifferentiated, elicited vine cells, as a viscous suspension or a gel or a substantially dry powder.

### Example 9

### Solutions

Solutions containing an aqueous phase A only, essentially based on demineralised water, perfume, glycerol, preservative and active substance: comminuted, encapsulated dedifferentiated, elicited vine cells, as a viscous suspension or a gel or a substantially dry powder, or a cell suspension as prepared by the first method.

### Example 10

### Milks

aqueous phase A: essentially based on deionised water

oleaginous phase B: oil+emulsifier+emollient

phase C: preservative+moisturising product

phase D: active substance: comminuted, encapsulated dedifferentiated, elicited vine cells, as a viscous suspension or a gel or a substantially dry powder, or a cell suspension as prepared by the first method.

In the examples given above relating to creams, gels or milks, the different phases A, B, C and D, in proportions which can vary according to the desired application, are mixed in a gentle manner, so as not to damage completely the coating layer encapsulating cells or cells aggregates.

The proportion of encapsulated UV-elicited dedifferentiated plant cells, especially vine cells, can vary from 0.1% by weight (as dry matter) up to 25% by weight, but preferably from 0.1% up to 5% by weight (as dry matter).

The invention is obviously not limited to the examples given above, and it is possible to produce the composition for topical use in other forms, such as oils, ointments, lacquers, colours (foundation, powder, lipstick, pencil, mascara, eye shadow), which also fall within the scope of the invention.

Moreover, the invention is not limited to vine cells and can be applied to other types of plant cells provided that they can be obtained in dedifferentiated form and are capable of undergoing elicitation resulting in an accumulation of secondary metabolites in an amount sufficient quantity to facilitate biological activity in topical use.

### Example 11

Example 1 was repeated using dedifferentiated plant cells originating from different plant species or mixtures of different plant species. In these examples, peel, seed, beans, roots, leaves, stems, buds, fruits, skin or cuticle were used in order to obtain encapsulated UV-elicited dedifferentiated plant cells

The following Table lists the plant species used:

| Example 11 | Plant species |
|---|---|
| A | Rosmarinus |
| B | Coffea |
| C | Cacao |
| D | Mungo |
| E | Colchicum |
| F | Jasminuna + Iris |
| G | Capsicum |
| H | Pilocarpus |
| I | Sequoia |
| J | Solanum |
| K | Chlorophytum |
| L | Gingko |
| M | digitalis |
| N | Salvia |
| O | Taxus |
| P | Papaver |
| Q | Salvia + rosmarinus |
| R | Roses |
| S | Tea |
| T | Betula |
| U | Grapevine + citrus + ginko |

The anti-radical and anti-oxidant activity of the product obtained according to Examples 11A to 11U was investigated in vitro. A SKINETHIC® reconstituted model epidermis was used, which enabled this activity to be revealed by the determination of malondialdehyde (MDA) after the induction thereof by ultraviolet B radiation.

For said test, plant cells prepared according to the first method or according to the second method have been used. Said cells were prepared one year before the test, so as to confirm the stability of the encapsulated UV-elicited dedifferentiated plant cells of the invention.

Said tests have shown that the encapsulated UV-elicited dedifferentiated plant cell of the invention had an antioxidant activity and an anti-radical effect just after preparation, as well as 3 months, 6 months and 12 months after their preparation.

What we claims is:

1. A cosmetic composition for topical application comprising at least
(a) a dispersion of at least encapsulated UV-elicited dedifferentiated plant cells, whereby said encapsulated UV-elicited dedifferentiated plant cells comprise vacuoles comprising at least stilbenes, vitamins, proteins and amino acids, the dry weight of stilbenes selected from the group consisting of resveratrol, catechine, astringine and piceide with respect to the dry weight of the encapsulated UV-elicited dedifferentiated plant cells before being communited is at least 0.1%, the cell being provided with a bi-lipidic membrane comprising at least C₁₂-C₂₀ fatty acids, cholesterol and ceramides, whereby said encapsulated UV-elicited dedifferentiated plant cells are at least partly encapsulated with glycosaminoglycans and whereby it said encapsulated UV-elicited dedifferentiated plant cells are uncommunited, whereby the cosmetic composition comprises from 0.1% by weight to 5% by weight of said encapsulated UV-elicited dedifferentiated plant cells; and
(b) a cosmetically acceptable excipient.

2. The composition of claim 1, in which the encapsulated UV-elicited dedifferentiated plant cells are dedifferentiated plant cells grown in suspension in vitro in a culture medium and UV-elicited in said culture medium for at least 7 to 30 days for synthesising stilbene into the cells and for storing stilbenes within the vacuoles of the UV-elicited dedifferentiated plant cells.

3. The composition of claim 1, in which the encapsulated UV-elicited dedifferentiated plant cells are dedifferentiated plant cells grown in suspension in vitro in a culture medium and UV-elicited in said culture medium for at least 7 to 30 days for synthesising stilbenes into the cells and for storing stilbenes within the vacuoles of the UV-elicited dedifferentiated plant cells, whereby the culture medium comprises at least one sugar and at least one additive selected from the group consisting of unsaturated C₁₂-C₂₀ fatty acid and pectin.

4. The composition of claim 3, in which the weight ratio additive selected from the group consisting of unsaturated C₁₂-C₂₀ fatty acid and pectin present in the culture medium/dedifferentiated plant cells growing in the culture medium expressed in dry form is comprised between 0.2:1 and 10:1.

5. The composition of claim 1, in which the encapsulated UV-elicited dedifferentiated plant cells are dedifferentiated plant cells grown in suspension in vitro in a culture medium and UV-elicited in said culture medium for at least 7 to 30 days for synthesising stilbene into the cells and for storing stilbenes within the vacuoles of the UV-elicited dedifferentiated plant cells, whereby the culture medium comprises at least sucrose and at least one additive selected from the group consisting of unsaturated C₁₂-C₂₀ fatty acid and pectin.

6. The composition of claim 5, in which the weight ratio additive selected from the group consisting of unsaturated C₁₂-C₂₀ fatty acid and pectin present in the culture medium/dedifferentiated plant cells growing in the culture medium expressed in dry form is comprised between 0.2:1 and 10:1.

7. The composition of claim 1, which comprises from 0.2% by weight to 2% by weight of said encapsulated UV-elicited dedifferentiated plant cells.

8. The composition of claim 1, which comprises encapsulated UV-elicited dedifferentiated vine cells.

9. The composition of claim 1, which comprises encapsulated UV-elicited dedifferentiated plant cell selected from the group species consisting of Salvia, Coleus, Rosmiarinus, Ginkgo, Cannabis, Coichicum, Gloriosa, Asparagus, Arganier, Wisteria, Medicago, Mungo, Erythrina, Oenothera, Papaver, Atropa, Datura, Solanum, Borago, Reseda, Amsonia, Catharantus, Pilocarpus, Digitalis, Coffea, Theobroma, Jasminum, Capsicum, Iris, vine, taxus, blue lotus, oriental cherry, sequoia, chlorophytum, Cacao, psoralea coryilfolia, vitex negundo, commiphora wighii, eucalyptus punctata, lavandula angustifolia, citrus limon, vanilla planifolia, marrubium vulgare, pilocarpus jaborandi, roses, hetula, tea, and mixtures thereof.

10. The composition of claim 1, said encapsulated UV-elicited dedifferentiated plant cells are at least partly encapsulated with glycosaminoglycans and at least one component selected from the group consisting of glycerol, ethyleneglycol, propyleneglycol, diethyleneglycol, butyleneglycol and mixtures thereof.

11. The composition of claim 10, in which the weight ratio component selected from the group consisting of glycerol, ethyleneglycol, propyleneglycol, diethyleneglycol, butyleneglycol and mixtures thereof/UV-elicited dedifferentiated plant cells is greater than 2:1.

12. The composition of claim 1, said encapsulated UV-elicited dedifferentiated plant cells are at least partly encapsulated with glycosaminoglycans and at least one component selected from the group consisting of glycerol, ethyleneglycol, propyleneglycol, diethyleneglycol, butyleneglycol and mixtures thereof, in which the weight ratio component selected from the group consisting of glycerol, ethyleneglycol, propyleneglycol, diethyleneglycol, butyleneglycol and mixtures thereof/UV-elicited dedifferentiated plant cells is comprised between 3:1 and 10:1.

13. The composition of claim 1, in which the encapsulated UV-elicited dedifferentiated plant cells are dedifferentiated plant cells grown in suspension in vitro in a culture medium, encapsulated at least with glycosaminoglycans and at least one component selected from the group consisting of glycerol, ethyleneglycol, propyleneglycol, diethyleneglycol, butyleneglycol and mixtures thereof before being UV-elicited in said culture medium for at least 7 to 30 days for synthesising stilbenes into the cells and for storing stilbene within the vacuoles of the UV-elicited dedifferentiated plant cells.

14. The composition of claim 1, in which said encapsulated UV-elicited dedifferentiated plant cells are at least partly encapsulated with glycosaminoglycan selected from the group consisting of Chondroitin and cosmetically acceptable salts thereof, heparin and cosmetically acceptable salts thereof. Heparitin and cosmetically acceptable salts thereof, Hyaluronic Acid and cosmetically acceptable salts thereof, Keratan and cosmetically acceptable salts thereof, and their mixtures.

15. The composition of claim 1, in which said encapsulated UV-elicited dedifferentiated plant cells are at least partly encapsulated with glycosaminoglycans and trehalose.

16. The composition of claim 1 which further comprises powder of communited lyophilized encapsulated UV-elicited dedifferentiated plant cells, whereby said encapsulated UV-elicited dedifferentiated plant cells comprise before being communited vacuoles comprising at least stilbenes, vitamins, proteins and amino acids, whereby the dry weight of stilbenes selected from the group consisting of resveratrol, catechine, astringine and piceide with respect to the dry weight of the encapsulated UV-elicited dedifferentiated plant cells before being communited is at least 0.1%, the encapsulated UV-elicited dedifferentiated plant cells before being communited being provided with a bi-lipidic membrane comprising at least C₁₂-C₂₀ fatty acids, cholesterol and ceramides, whereby said encapsulated UV-elicited dedifferentiated plant cells before being communited are at least partly encapsulated with glycosaminoglycans, and in which the dry weight ratio powder of communited lyophilised encapsulated UV-elicited dedifferentiated plant cells/non communited encapsulated UV-elicited dedifferentiated plant cells is comprised between 1:10 and 10:1.

17. The composition of claim 16, in which said powder of communited lyophilised encapsulated UV-elicited dedifferentiated plant cells is prepared from dedifferentiated plant cells at least partly encapsulated with glycosaminoglycan selected from the group consisting of Chondroitin and cosmetically acceptable salts thereof, heparin and cosmetically acceptable salts thereof, Heparitin and cosmetically acceptable salts thereof, Hyaluronic Acid and cosmetically acceptable salts thereof, Keratan and cosmetically acceptable salts thereof, and their mixtures.

18. The composition of claim 16, in which the powder of communited lyophilized encapsulated UV-elicited dedifferentiated plant cells is a powder of communited UV-elicited dedifferentiated plant cells encapsulated at least with glycosaminoglycans and trehalose.

19. A cosmetic composition for topical application comprising at least:
(a) a powder of communited lyophilized encapsulated UV-elicited dedifferentiated plant cells, whereby said encapsulated UV-elicited dedifferentiated plant cells comprise before being communited vacuoles comprising at least stilbenes vitamins, proteins and amino acids, whereby the dry weight of stilbenes selected from the group consisting of resveratrol, catechine, astringine and piceide with respect to the dry weight of the encapsulated UV-elicited dedifferentiated plant cells before being communited is at least 0.1%, the encapsulated UV-elicited dedifferentiated plant cells before being communited being provided with a bi-lipidic membrane comprising at least C₁₂-C₂₀ fatty acids, cholesterol and ceramides, whereby said encapsulated UV-elicited dedifferentiated plant cells before being communited are at least partly encapsulated with glycosaminoglycans, whereby the cosmetic composition comprises from 0.1% by weight to 5% by weight of said powder of communited lyophilized encapsulated UV-elicited dedifferentiated plant cells; and
(b) a cosmetically acceptable excipient.

20. The composition of claim 19, in which said powder of communited lyophilised encapsulated UV-elicited dedifferentiated plant cells is prepared from dedifferentiated plant cells at least partly encapsulated with glycosaminoglycan selected from the group consisting of Chondroitin and cosmetically acceptable salts thereof, heparin and cosmetically acceptable salts thereof, Heparitin and cosmetically acceptable salts thereof, Hyaluronic Acid and cosmetically acceptable salts thereof, Keratan and cosmetically acceptable salts thereof, and their mixture.

21. The composition of claim 19, in which said powder has an average particle size of less than 10 μm.

22. The composition of claim 19, in which said powder has an average particle size of less than 1 μm.

23. The composition of claim 17, which comprises powder of communited lyophilised encapsulated UV-elicited dedifferentiated vine cells.

24. The composition of claim 17, in which the powder of comminuted lyophilised encapsulated UV-elicited dedifferentiated cells is derived from the culture of dedifferentiated plant cells, which are elicited and then dried, of at least one species selected from the group consisting of Salvia, Coleus, Rosmarinus, Gingko, Cannabis, Colchicum, Gloriosa, Asparagus, Arganier, Wisteria, Medicago, Mungo, Erythrina, Oenothera, Papaver, Atropa, Datura, Solanum, Borago, Reseda, Amsonia, Catharantus, Pilocarpus, Digitalis, Coffea, Theobroma, Jasminum, Capsicum, Iris, vine, taxus, blue lotus, oriental cherry, sequoia, chlorophytum, Cacao, psoralea coryilfolia, vitex negundo, commiphora wighii, eucalyptus punctata, lavandula angustifolia, citrus limon, vanilla planifolia, marrubium vulgare, pilocarpus jaborandi, roses, betula, tea and mixtures of cells of such species.

25. The composition of claim 17, in which the powder of communited lyophilized encapsulated UV-elicited dedifferentiated plant cells is a powder of communited UV-elicited dedifferentiated plant cells encapsulated at least with glycosaminoglycans and trehalose.

26. A method of preparing a cosmetic composition for topical application comprising at least:
(a) a dispersion of at least encapsulated UV-elicited dedifferentiated plant cells, whereby said encapsulated UV-elicited dedifferentiated plant cells comprise vacuoles comprising at least stilbenes, vitamins, proteins and amino acids, whereby the dry weight of stilbenes selected from the group consisting of resveratrol, catechine, astringine and piceide with respect to the dry weight of the encapsulated UV-elicited dedifferentiated plant cells before being communited is at least 0.1%, the cell being provided with a bi-lipidic membrane comprising at least C₁₂-C₂₀ fatty acids, cholesterol and ceramides, whereby said encapsulated UV-elicited dedifferentiated plant cells are at least partly encapsulated with glycosaminoglycans and whereby said encapsulated UV-elicited dedifferentiated plant cells are uncommunited, whereby the cosmetic composition comprises from 0.1% by weight to 5% by weight of said encapsulated UV-elicited dedifferentiated plant cells, and
(b) a cosmetically acceptable excipient,
said method comprising at least the following steps:
step 1 growing, in vitro, dedifferentiated plant cells in an agar-agar containing culture medium in a sterile atmosphere at a temperature comprised between 10° C. and 35° C., so as to form a culture of dedifferentiated plant cells;
step 2: placing dedifferentiated plant cells from the culture of step 1 in suspension and mixing said dedifferentiated plant cells in a liquid sucrose containing culture medium enriched with at least 0.5% by weight of glycosaminoglycan with respect to the dry weight of sucrose;
step 3: growing the dedifferentiated plant cells in the glycosaminoglycan enriched sucrose liquid culture medium, while submitting the dedifferentiated plant cells to an elicitation by UV light with a wave length comprised between 200 nm and 300 nm, at a temperature comprised between 10° C. and 35° C. for 7 to 30 days, so as to produce encapsulated UV-elicited dedifferentiated plant cells, whereby said encapsulated UV-elicited dedifferentiated plant cells comprise vacuoles comprising at least stilbenes, vitamins, proteins and amino acids, whereby the dry weight of stilbenes selected from the group consisting of resveratrol, catechine, astringine and piceide with respect to the dry weight of the encapsulated UV-elicited dedifferentiated plant cells before being communited is at least 0.1%, the cell being provided with a bi-lipidic membrane comprising at least C₁₂-C₂₀ fatty acids, cholesterol and ceramides, whereby said encapsulated UV-elicited dedifferentiated plant cells are at least partly encapsulated with glycosaminoglycans;
step 4: recovering the encapsulated UV-elicited dedifferentiated plant cells from the glycosaminoglycan enriched sucrose liquid culture medium; and
step 5: mixing the encapsulated UV-elicited dedifferentiated plant cells with cosmetically acceptable excipients at a temperature below 35° C., so as to prepare a cosmetic composition comprising from 0.1% by weight to 5% by weight of said encapsulated UV-elicited dedifferentiated plant cells.

27. The method of claim 26, including the step of adding at least one component selected from the group consisting of glycerol, ethyleneglycol, propyleneglycol, diethyleneglycol, butyleneglycol and mixtures thereof to the glycosaminoglycan enriched sucrose liquid culture medium, such adding step taking place at a time selected from the group consisting of: (a) before the elicitation of the dedifferentiated plant cells by UV light with a wave length comprised between 200 nm and 300 nm, (b) during the elicitation of the dedifferentiated plant cells by UV light with a wave length comprised between 200 nm and 300 nm, (c) after the elicitation of the dedifferentiated plant cells by UV light with a wave length comprised between 200 nm and 300 nm, and combinations thereof, so as to form encapsulated UV-elicited dedifferentiated plant cells encapsulated at least with glycosaminoglycan and at least one component selected from the group consisting of glycerol, ethyleneglycol, propyleneglycol, diethyleneglycol, butyleneglycol and mixtures thereof.

28. The method of claim 26, including the step of adding at least one component selected from the group consisting of glycerol, ethyleneglycol, propyleneglycol, diethyleneglycol, butyleneglycol and mixtures thereof to the glycosaminoglycan enriched sucrose liquid culture medium, at a time at least before the elicitation of the dedifferentiated plant cells by UV light with a wave length comprised between 200 nm and 300 nm, so as to form encapsulated UV-elicited dedifferentiated plant cells encapsulated at least with glycosaminoglycan and at least one component selected from the group consisting of glycerol, ethyleneglycol, propyleneglycol, diethyleneglycol, butyleneglycol and mixtures thereof.

29. The method of claim 26, in which in step 2, the dedifferentiated plant cells from the culture of step 1 are put in suspension and mixed in a liquid sucrose containing culture medium enriched with at least 0.5% by weight of glycosaminoglycan selected from the group consisting of Chondroitin and cosmetically acceptable salts thereof, heparin and cosmetically acceptable salts thereof, Heparitin and cosmetically acceptable salts thereof, Hyaluronic Acid and cosmetically acceptable salts thereof, Keratan and cosmetically acceptable salts thereof, and their mixtures.

30. The method of claim 26, in which, at least for a part of step 3, trehalose is added to the sucrose liquid culture medium, whereby the dedifferentiated plant cells are growth at least partly in a glycosaminoglycan and trehalose enriched sucrose liquid culture medium, while being submitted to an elicitation by UV light with a wave length comprised between 200 nm and 300 nm.

31. The method of claim 30, wherein, at least for a latest stage of step 3, trehalose is added to the sucrose liquid culture medium, whereby the dedifferentiated plant cells are grown at least partly in a glycosaminoglycan and trehalose enriched sucrose liquid culture medium, while being submitted to an elicitation by UV light with a wave length comprised between 200 nm and 300 nm.

32. A method of preparing a cosmetic composition for topical application comprising at least:
(a) a powder of communited lyophilized encapsulated UV-elicited dedifferentiated plant cells, whereby said encapsulated UV-elicited dedifferentiated plant cells comprise before being communited vacuoles comprising at least stilbenes, vitamins, proteins and amino acids, whereby the dry weight of stilbenes selected from the group consisting of resveratrol, catechine, astringine and piceide with respect to the dry weight of the encapsulated UV-elicited dedifferentiated plant cells before being communited is at least 0.1%, the encapsulated UV-elicited dedifferentiated plant cells before being communited being provided with a bi-lipidic membrane comprising at least C₁₂-C₂₀ fatty acids, cholesterol and ceramides, whereby said encapsulated UV-elicited dedifferentiated plant cells before being communited are at least partly encapsulated with glycosaminoglycans whereby the cosmetic composition comprises from 0.1% by weight to 5% by weight of said powder of communited lyophilized encapsulated UV-elicited dedifferentiated plant cells, and
(b) a cosmetically acceptable excipient,
said method comprising at least the following steps:
step 1: growing dedifferentiated plant cells in vitro in an agar-agar containing culture medium in a sterile atmosphere at a temperature comprised between 10° C. and 35° C., so as to form a culture of dedifferentiated plant cells,
step 2: placing the dedifferentiated plant cells from the culture of step 1 in suspension and mixing said dedifferentiated plant cells in a liquid sucrose containing culture medium enriched with at least 0.5% by weight of glycosaminoglycan with respect to the dry weight of sucrose;
step 3: growing said dedifferentiated plant cells in the glycosaminoglycan enriched sucrose liquid culture medium, while submitting said dedifferentiated plant cells to an elicitation by UV light with a wave length comprised between 200 nm and 300 nm, at a temperature comprised between 10° C. and 35° C. for 7 to 30 days, so as to produce encapsulated UV-elicited dedifferentiated plant cells, whereby said encapsulated UV-elicited dedifferentiated plant cells comprise vacuoles comprising at least stilbenes, vitamins, proteins and amino acids, whereby the dry weight of stilbenes selected from the group consisting of resveratrol, catechine, astringine and piceide with respect to the dry weight of the encapsulated UV-elicited dedifferentiated plant cells before being communited is at least 0.1%, the cell being provided with a bi-lipidic membrane comprising at least C₁₂-C₂₀ fatty acids, cholesterol and ceramides, whereby said encapsulated UV-elicited dedifferentiated plant cells are at least partly encapsulated with glycosaminoglycans;
step 4: recovering the encapsulated UV-elicited dedifferentiated plant cells from the glycosaminoglycan enriched sucrose liquid culture medium;
step 5: lyophilising and comminuting the recovered encapsulated UV-elicited dedifferentiated plant cells in a powder; and
step 6: mixing the powder of comminuted lyophilised UV-elicited dedifferentiated plant cells with cosmetically acceptable excipients at a temperature below 35° C., so as to prepare a cosmetic composition comprising from 0.1% by weight to 5% by weight of said powder of comminuted lyophilised UV-elicited dedifferentiated plant cells.

33. The method of claim 32, in which at least one component selected from the group consisting of glycerol, ethyleneglycol, propyleneglycol, diethyleneglycol, butyleneglycol and mixtures thereof is added to the glycosaminoglycan enriched sucrose liquid culture medium, at least at a time selected from the group consisting of: (a) before the elicitation of the dedifferentiated plant cells by UV light with a wave length comprised between 200 nm and 300 nm, (b) during the elicitation of the dedifferentiated plant cells by UV light with a wave length comprised between 200 nm and 300 nm, (c) after the elicitation of the dedifferentiated plant cells by UV light with a wave length comprised between 200 nm and 300 nm, and combinations thereof, so as to form encapsulated UV-elicited dedifferentiated plant cells encapsulated at least with glycosaminoglycan and at least one component selected from the group consisting of glycerol, ethyleneglycol, propyleneglycol, diethyleneglycol, butyleneglycol and mixtures thereof.

34. The method of claim 32, in which at least one component selected from the group consisting of glycerol, ethyleneglycol, propyleneglycol, diethyleneglycol, butyleneglycol and mixtures thereof is added to the glycosaminoglycan enriched sucrose liquid culture medium, at least before the elicitation of the dedifferentiated plant cells by UV light with a wave length comprised between 200 nm and 300 nm, so as to form encapsulated UV-elicited dedifferentiated plant cells encapsulated at least with glycosaminoglycan and at least one component selected from the group consisting of glycerol ethyleneglycol, propyleneglycol, diethyleneglycol, butyleneglycol and mixtures thereof.

35. The method of claim 32, in which in step 2, the dedifferentiated plant cells from the culture of step 1 are put in suspension and mixed in a liquid sucrose containing culture medium enriched with at least 0.5% by weight of glycosaminoglycan selected from the group consisting of Chondroitin and cosmetically acceptable salts thereof heparin and cosmetically acceptable salts thereof, Heparitin and cosmetically acceptable salts thereof, Hyaluronic Acid and cosmetically acceptable salts thereof, Keratan and cosmetically acceptable salts thereof, and their mixtures.

36. The method of claim 32, in which at least for a part of step 3, trehalose is added to the sucrose liquid culture medium, whereby the dedifferentiated plant cells are growth at least partly in a glycosaminoglycan and trehalose enriched sucrose liquid culture medium, while being submitted to an elicitation by UV light with a wave length comprised between 200 nm and 300 nm.

37. The method of claim 36, at least for a latest stage of step 3, trehalose is added to the sucrose liquid culture medium, whereby the dedifferentiated plant cells are growth at least partly in a glycosaminoglycan and trehalose enriched sucrose liquid culture medium, while being submitted to an elicitation by UV light with a wave length comprised between 200 nm and 300 nm.
